(19) United States
(12) Patent Application Publication
(10) Pub. No.: US 2024/0018227 A1
(43) Pub. Date: Jan. 18, 2024

(54) COMPOSITIONS AND METHODS FOR TREATING DISORDERS CHARACTERIZED WITH TGF-BETA ACTIVITY

(71) Applicant: THE JOHNS HOPKINS UNIVERSITY, Baltimore, MD (US)

(72) Inventor: Xu Cao, Baltimore, MD (US)

(21) Appl. No.: 18/033,746

(22) PCT Filed: Oct. 27, 2021
(86) PCT No.: PCT/US2021/056862

§ 371 Date: Apr. 25, 2023

Related U.S. Application Data
(60) Provisional application No. 63/106,706, filed on Oct. 28, 2020.

(51) Int. Cl. C07K 16/22; A61P 19/04; A61P 25/00

(52) U.S. Cl. CPC C07K 16/22; A61P 19/04; A61P 25/00; C07K 2317/76; A61K 2039/505

(57) ABSTRACT

In Provided herein are compositions and methods directed to treating, delaying progression of, or ameliorating symptoms related to disorders characterized with TGF-β activity (e.g., disorders characterized with aberrant cartilage formation and/or osteoclast resorption (e.g., ankylosing spondylitis)) (e.g., spinal cord injuries) (e.g., fibrotic scar formation following spinal cord injury) through inhibition of TGF-β activity (e.g., thereby hindering and/or inhibiting aberrant cartilage formation and/or osteoclast resorption within joints, muscles, tendons, ligaments, connective tissue, and/or bones experiencing or at risk of experiencing ankylosis) (e.g., thereby inhibiting fibrotic scar formation following spinal cord injury).

This invention was made with government support under AR071432 awarded by the National Institutes of Health. The government has certain rights in the invention.

### FIELD OF THE INVENTION

Provided herein are compositions and methods directed to treating, delaying progression of, or ameliorating symptoms related to disorders characterized with TGF-β activity (e.g., disorders characterized with aberrant cartilage formation and/or osteoclast resorption (e.g., ankylosing spondylitis)) (e.g., spinal cord injuries) (e.g., fibrotic scar formation following spinal cord injury) through inhibition of TGF-β activity (e.g., thereby hindering and/or inhibiting aberrant cartilage formation and/or osteoclast resorption within joints, muscles, tendons, ligaments, connective tissue, and/or bones experiencing or at risk of experiencing ankylosis) (e.g., thereby inhibiting fibrotic scar formation following spinal cord injury).

### INTRODUCTION

Ankylosing spondylitis (AS) is an autoimmune disease that mainly affects spine and sacroiliac joint with a progressive increase in stiffness of soft tissues including ligaments, as a common type of spondyloarthritis. The prevalence rates of AS is between 0.1% to 0.32% worldwide, with nearly 1.3-1.6 million patients in Europe and 4.6-5.0 million patients in Asia. AS is more common within North America with reported prevalence to be 31.9 per 10000 among population (see, Dean LE, et al., Rheumatology (Oxford) 53, 650-657 (2014)). Related inflammation often leads to calcification, bone formation, accompanied with bone destructive lesions, resulting in the fusion of the spine, loss of flexibility and chronic back pain.

Significant progress in understanding the cause of this autoimmune disease and its treatment for the inflammation has been achieved in the last decade (see, Zhu W, et al. Bone Res 7, 22 (2019)). Immune cells and innate cytokines have been shown crucial in the pathogenesis of AS, especially human leukocyte antigen (HLA)-B27 and the interleukin-23/17 axis. HLA-B27 has been considered as the major genetic risk factor in AS since its discovery (see, Brewerton D A, et al., Lancet 1, 904-907 (1973)). However, the progress in autoimmune studies and anti-inflammatory treatments have not led to low ankylosing, largely due to limited understanding of how inflammation induces fusion of axial joints (see, Zhu W, et al. Ankylosing spondylitis: etiology, pathogenesis, and treatments. Bone Res 7, 22 (2019); Lories R J. Best Pract Res Clin Rheumatol 32, 331-341 (2018)). Spinal cord injuries (SCI) can cause permanent paralysis at and distal to the injury site, with sensorimotor dysfunction due to failed recovery from axonal and neuronal damage. The World Health Organization estimates that 250,000 to 500,000 people globally will experience some form of SCI every year^{1,2}. Most patients affected by SCI are young, eligible members of the workforce, and thus experience devastating physical, psychosocial, and financial effects of long-term disability³. SCI typically leads to formation of scar tissue with glial and fibrotic components that limit the restoration of damaged spinal circuitry affecting sensory, motor, and autonomous functions below the injury site⁴⁻⁷. Whereas scar-forming astrocytes have been studied extensively, much less attention has been given to the fibrotic, non-glial component of scar^{8,9}. The fibrotic component of scar consists of collagen, fibronectin, and fibroblasts that each present either a physical or molecular obstacle to the regeneration of severed axons^{8,9}. Scar formation is directly related to the degree of inflammation, as well as the number of macrophages at the site of injury¹¹. However, the role of macrophages in fibrotic scar formation after SCI is poorly understood.

Thus, there is a critical need for improved understanding of the relationship between inflammation and AS and SCI. In addition, there is a critical need for improved treatments for disorders characterized with AS and SCI.

The present invention addresses these needs.

### SUMMARY OF THE INVENTION

Anti-TNF-α antibody treatment effectively reduces active inflammation lesions up to 80% in 6-24 months. The expectation is that the progression of AS should also be halted, but no significant efficacy in reduction of spine ossification and fusion has been detected, not even as significant as with NSAID treatment (see, Schett G, et al., Best Pract Res Clin Rheumatol 24, 363-371 (2010); Poddubnyy D, et al., Expert Opin Biol Ther 13, 1599-1611 (2013)). Indeed, overexpression human TNF-α in mice is associated with the development of sacroiliitis (see, Keffer J, et al. EMBO J 10, 4025-4031 (1991)). Radiographic progression in AS patients with infliximab treatment suggests that bony structural progression was independent of TNF-α, different from other inflammatory rheumatic diseases such as rheumatoid arthritis and psoriatic arthritis (see, Uderhardt S, et al Ann Rheum Dis 69, 592-597(2010)). MRI data has shown that new bone formation in syndesmophytes is three-times more in the active inflammation area than no active inflammation, and syndesmophytes occur in sites with previous inflammation, but not persistent inflammation (see, Baraliakos X, et al., Arthritis Res Ther 10, R104 (2008)). Long term anti-TNFα therapy using different anti-TNF-α a agents have resulted in improvement in spinal inflammation in AS patients but there were no signs of inhibition of structural damage progression (see, van der Heijde D, et al. Arthritis Rheum 58, 3063-3070 (2008); van der Heijde D, et al, Arthritis Rheum 58, 1324-1331 (2008); Rudwaleit M, et al., Ann Rheum Dis 64, 1305-1310 (2005); Baraliakos X, et al. Arthritis Rheum 53, 856-863 (2005)). Thus, there does not appear to be a simple relationship between inhibition of inflammation and bony progression, suggesting inflammation does not directly trigger new bone formation.

The development of ankylosis appears to be uncoupled from inflammation (see, Maksymowych W P. Nat Rev Rheumatol 6, 75-81 (2010)). In vivo studies using well studied DBA/1 mice for AS have demonstrated the uncoupling of inflammation and joint remodeling in spondylarthritis (see, Lories RJU, et al., Arthritis Rheum 56, 489-497 (2007)). Patients having undergone anti-inflammation treatment still developed syndesmophytes at sites where no inflammation had been detected by MRI (see, van der Heijde D, et al. Ann Rheum Dis 71, 369-373 (2012)). Moreover, MRI detected inflammation in a vertebral unit just slightly increases the possibility to form a new syndesmophyte, and would not affect the growth of already existing syndesmophyte (see, Francois RJ, et al., Arthritis Rheum 43, 2011-2024 (2000)). It appears the inflammation is a precondition of bony formation in AS.

Systematic study of specimens from AS patients revealed that there was woven bone within the sacroiliac ligament (see, Francois RJ, et al., Arthritis Rheum 43, 2011-2024 (2000)), suggesting AS is not merely enthesitis. Besides excessive bone formation in AS patients, the prevalence rate of osteoporosis in AS patients was between 9.5% and 40% (see, Magrey M, Khan M A. Curr Rheumatol Rep 12, 332-336 (2010); Hinze A M, et al., Curr Treatm Opt Rheumatol 2, 271-282 (2016); Davey-Ranasinghe N, et al., Curr Opin Rheumatol 509-516 (2013); Ulu MA, et al., Chin Med J 127, 2740-2747 (2014); Beek KJ, et al. J Bone Miner Res 34, 1041-1048 (2019)). There were significantly more osteoclast in AS patients at bone resorption sites (see, Appel H, et al. Arthritis Rheum 54, 1805-1813 (2006); Appel H, et al, Arthritis Rheum 54, 2845-2851 (2006)), and the level of osteoclasts has no relationship with the inflammation activity (see, Appel H, et al, Arthritis Rheum 54, 2845-2851 (2006)). Bisphosphonates are well known drugs which not only can inhibit osteoclasts, but also improve osteoporotic bone density measurements and prevent fractures (see, Van Offel J F, et al., Clin Exp Rheumatol 19, 13-20 (2001)). Patients with AS treated with bisphosphonate achieved similar results regarding reduction of disease activity and benefit to bone mineral density (BMD) (see, Viapiana O, et al. Rheumatology (Oxford) 53, 90-94 (2014)). Using bisphosphonates for AS treatment was effective on ameliorating either clinical index or radiological progression in AS patients (see, Singh R, Menon Y, et al., Arthritis Rheum 48, (2003); Maksymowych WP, et al. J Rheumatol 28, 144-155 (2001); Toussirot E, Wendling D. Curr Opin Rheumatol 19, 340-345 (2007); Haibel H, et al., Rheumatology (Oxford) 42, 1018-1020 (2003)), no matter treatment was adopted in a short term (see, Singh R, Menon Y, et al., Arthritis Rheum 48, (2003)) or in an intermittent way (see, Maksymowych WP, et al. J Rheumatol 28, 144-155 (2001)). Therefore, bony formation in AS does not just originate from enthesitis as osteoclast played an important role in structural disease progression. Indeed, the underlying mechanism is still waiting to be expounded.

Acquired heterotopic ossification (HO) is a painful and debilitating disease characterized by extra skeletal bone formation after injury. It has been reported that transforming growth factor—β (TGF-β) inhibitor initiates and promotes HO during four different stages: 1) inflammation, 2) chondrogenesis, 3) osteogenesis, and 4) maturation (see, Wang X, et al. Nat Commun 9, 551 (2018)). Previous experiments determined that chondrocyte differentiation and cartilage formation is an intermediate phase prior ossification in HO. Importantly, osteoclast resorption of calcified cartilage activates excessive active TGF-β that recruits mesenchymal stromal/progenitor cells in the HO microenvironment for the ectopic bone formation (see, Wang X, et al. Nat Commun 9, 551 (2018)). Even at mature stage, active TGF-β are continuously activated by osteoclast resorption drives progression of HO. Interestingly, serum level of TGF-β significant elevated in AS patients and remained at high level regardless the activity of disease (see, Taylan A, et al. Rheumatol Int 32, 2511-2515 (2012)). Anti-TNF-α treatment improved the AS functional and disease activity index with no effect on the serum level of active TGF-β (see, Taylan A, et al. Rheumatol Int 32, 2511-2515 (2012); Visvanathan S, et al., Ann Rheum Dis 68, 175-182 (2009)). Moreover, immunohistological study of sacroiliac joints biopsies also found advanced AS patients with high level of active TGF-β (see, Tang Y, et al. Nat Med 15, 757-765 (2009)). The pathogenesis of characterized acquired HO indicates spine bony fusion could undergo a similar pathological process of acquired HO (see, Wang X, et al. Nat Commun 9, 551 (2018)).

Experiments conducted during the course of developing embodiments for the present invention found chondrocyte differentiation and cartilage formation in the spine ligaments at early stage of AS patients. Large number of giant osteoclasts were found at the bony surface with elevated active TGF-β, which promoted recruitment of Osterix⁺ osteoprogenitors for ossification of spine fusion. Such results demonstrate that the progression AS undergoes similar stages with the pathogenesis of acquired HO: inflammation, chondrogenesis, osteogenesis, and maturation. Therefore, such results indicate inhibition of cartilage formation and osteoclast resorption activity as an effective therapy for treatment, prevention or amelioration of conditions characterized with aberrant chondrocyte differentiation and cartilage formation (e.g., AS).

After SCI, macrophages are recruited to the lesion center as a source of anti-inflammatory factors to promote tissue repair and provide a source of inflammatory cytokines to fuel secondary injury¹²⁻¹⁴. Hematogenous macrophages can recruit fibroblasts to the injury site to form fibrotic scar⁶. Interestingly, perivascular fibroblasts have been shown to be the primary source of collagen and fibronectin in kidney fibrosis¹⁵. Emerging evidence reveals that fibrotic scar is involved in a subset of perivascular cells for regaining tissue integrity, this subset cells can also differentiation to fibroblast-like cells in secretion of extracellular matrix^{16,17}. The specific cytokine in recruitment of these pericytes is unknown. Transforming growth factor-βs (TGF-β), a family of 3 multifunctional peptide growth factors, are only present in mammals and involved in tissue remodeling, repair, and homeostasis¹⁸⁻²⁰. Importantly, many different diseases are associated with aberrant activation of TGF-β, such as fibrosis of the skin, kidneys, lungs, and liver²¹⁻²³, metastasis of tumors^{24,25}, heterotopic ossificatio²⁶, and osteoarthritis^{27,28}. Particularly, high levels of TGF-β1 are also observed during the progression of SCI²⁹.

In fibrosis of various tissues, elevated active TGF-β induces fibroblast differentiation of mesenchymal stromal/stem cells (MSCs)³⁰⁻³². It is also possible that elevated active TGF-β at the SCI site recruits MSCs to induce fibroblast differentiation for fibrotic scar formation. Experiments conducted during the course of developing embodiments for the present invention investigated the role of active TGF-β in fibrotic scar formation in SCI. It was found that active TGF-β was significantly elevated at the injury site after SCI to induce fibroblast differentiation of MSCs. Inhibition of overactive TGF-β signaling significantly reduced fibrotic scar formation and improved the recovery from SCI in mice. Importantly, as SCI in neonatal mice leads to scar-free healing³³, it was further found that neonatal mice did not have active TGF-β at the injury site and recovered completely from SCI.

Accordingly, the present invention provides compositions and methods directed to treating, delaying progression of, or ameliorating symptoms related to disorders characterized with TGF-β activity (e.g., disorders characterized with aberrant cartilage formation and/or osteoclast resorption (e.g., ankylosing spondylitis)) (e.g., spinal cord injuries) (e.g., fibrotic scar formation following spinal cord injury) through inhibition of TGF-β activity (e.g., thereby hindering and/or inhibiting aberrant cartilage formation and/or osteoclast resorption within joints, muscles, tendons, ligaments, connective tissue, and/or bones experiencing or at risk of experiencing ankylosis) (e.g., thereby inhibiting fibrotic scar formation following spinal cord injury).

In certain embodiments, the present invention provides methods for treating, delaying progression of, or reducing the severity of disorders characterized with aberrant cartilage formation and/or osteoclast resorption (e.g., ankylosing spondylitis) through hindering and/or inhibiting aberrant cartilage formation and/or osteoclast resorption within joints, muscles, tendons, ligaments, connective tissue, and/or bones experiencing or at risk of experiencing ankylosis. Such methods are not limited to particular type or manner of treating, delaying progression of, or reducing the severity of disorders characterized with aberrant cartilage formation and/or osteoclast resorption (e.g., ankylosing spondylitis) through hindering and/or inhibiting aberrant cartilage formation and/or osteoclast resorption within joints, muscles, tendons, ligaments, connective tissue, and/or bones experiencing or at risk of experiencing ankylosis.

In some embodiments, the present invention provides methods for treating, delaying progression of, or reducing the severity of disorders characterized with aberrant cartilage formation and/or osteoclast resorption (e.g., ankylosing spondylitis) through hindering and/or inhibiting aberrant cartilage formation and/or osteoclast resorption within joints, muscles, tendons, ligaments, connective tissue, and/or bones experiencing or at risk of experiencing ankylosis comprising administering to a subject in need thereof a therapeutically effective amount of an agent capable of hindering and/or inhibiting cartilage formation and/or osteoclast resorption within joints, muscles, tendons, ligaments, connective tissue, and/or bones experiencing or at risk of experiencing ankylosis.

In some embodiments, such administration results in one or more of the following within joints, muscles, tendons, ligaments, connective tissue, and/or bones experiencing or at risk of experiencing ankylosis: inhibition of cartilage formation; inhibition of osteoclast resorption activity; reduction and/or inhibition of TGF-β expression and/or activity; reduction and/or inhibition of pSmad1/5/8 signaling; reduction and/or inhibition of pSmad2/3 signaling; inhibition and/or hindering of chondrogenesis; inhibition and/or hindering of chondrocyte differentiation; prevention ankylosing spondylitis development; and reduction in the severity of ankylosing spondylitis.

Such methods are not limited to particular types or kinds of disorders characterized with aberrant cartilage formation and/or osteoclast resorption. In some embodiments, the disorder characterized with aberrant cartilage formation and/or osteoclast resorption is ankylosing spondylitis (AS). In some embodiments, the disorder characterized with aberrant cartilage formation and/or osteoclast resorption is heterotopic ossification. In some embodiments, the disorder characterized with aberrant cartilage formation and/or osteoclast resorption is rheumatoid arthritis, metastatic bone disease and/or Paget disease. Such embodiments are not limited to a particular type of agent capable hindering and/or inhibiting aberrant cartilage formation and/or osteoclast resorption within joints, muscles, tendons, ligaments, connective tissue, and/or bones experiencing or at risk of experiencing ankylosis (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In some embodiments, the agent is capable of one or more of the following within joints, muscles, tendons, ligaments, connective tissue, and/or bones experiencing or at risk of experiencing ankylosis: inhibition of cartilage formation; inhibition of osteoclast resorption activity; reduction and/or inhibition of TGF-β expression and/or activity; reduction and/or inhibition of pSmad1/5/8 signaling; reduction and/or inhibition of pSmad2/3 signaling; inhibition and/or hindering of chondrogenesis; inhibition and/or hindering of chondrocyte differentiation; prevention ankylosing spondylitis development; and reduction in the severity of ankylosing spondylitis.

In some embodiments, the agent is a TGF-β inhibitor. As used herein, the term TGF-β inhibitor means a small molecule, antibody or functional portion or fragment thereof, proteins, peptides, siRNAs, antagonists, agonists, compounds, or nucleotide constructs which either reversibly or irreversibly bind TGF-β and prevent its binding to a TGF-β receptor on a cell or tissue in a subject. The term can also mean a small molecule, antibody or functional portion or fragment thereof, proteins, peptides, siRNAs, antagonists, agonists, compounds, or nucleotide constructs which either reversibly or irreversibly bind TGF-β receptors in an antagonistic manner such that TGF-β and its analogs or derivatives cannot stimulate the TGF-β receptors in cells and tissues in a subject.

Examples of TGF-β inhibitors include, for example, antibodies such as (1D11) (see, Wang, et al., J. Clin. Invest. 2018, 128(2):846-860), Fresolimumab, Galunisertib, Lerdelimumab (CAT-152), Metelimumab (CAT-192), GC-1008, SR-2F, and 2G7, small molecule inhibitors such as GW788388 (4-{443-[3-(Pyridin-2-yl)-1H-pyrazol-4-yl]-pyridin-2-yl}-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxo1-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1 and Lucanix, soluble antagonists such as TGFβ-BRII:Fc, and Betaglycan/TGFβRIII. In some embodiments, the TGF-β inhibitor is TGF-β type 1 receptor inhibitor (TβR1I) (see, Qin, et al., Annals. N.Y. Acad. Sci. 1433:29-40).

In some embodiments, the agent is a retinoic acid agonist (e.g., a retinoic acid a agonist; a retinoic acid y agonist) (e.g., see, Shimono, et al., J. Orthop. Res. 28:217-277, 2010; Pavey, et al., Bone 90 (2016), 159-167; Uchibe, et al., J. Orthop. Res. 35:1096-1105, 2017; Shimono, et al., Nat. Med. 2011 April 17(4): 454-460); WO2016144976A9). RAR agonists may include any agent that activates RAR or sustains retinoic acid so that its activity at RAR increases. This includes both substances that initiate a physiological response when combined with a receptor, as well as substances that prevent the catabolism (or breakdown) of retinoids (for example, retinoic acid), allowing the signal from retinoic acid itself to increase. In some embodiments, the retinoic acid a agonist is selected from ATRA, AM580, AM80 (tamibarotene), BMS753, BD4, AC-93253, AR7, and NRX195183. In some embodiments, the retinoic acid y agonist is selected from Palovartene, CD2665, MM11253, 7a, NRX204647.

In some embodiments, the agent is an angiotensin II receptor antagonist (e.g., losartan, valsartan). An angiotensin II receptor antagonist is an angiotensin receptor blocker that is known to suppress the TGF-β signaling cascade.

In any of the described embodiments, the agent is formulated to be administered in any desirable manner (e.g., locally, orally, systemically, intravenously, intraarterially, subcutaneously, or intrathecally).

In certain embodiments of the invention, combination treatment with the agent capable of hindering and/or inhibiting aberrant cartilage formation and/or osteoclast resorption within joints, muscles, tendons, ligaments, connective tissue, and/or bones experiencing or at risk of experiencing ankylosis and a course of a drug known for treating disorders characterized with aberrant cartilage formation and/or osteoclast resorption (e.g., a drug known for treating treating ankylosing spondylitis).

The invention also provides pharmaceutical compositions comprising the agent capable of hindering and/or inhibiting aberrant cartilage formation and/or osteoclast resorption within joints, muscles, tendons, ligaments, connective tissue, and/or bones experiencing or at risk of experiencing ankylosis in a pharmaceutically acceptable carrier.

Such methods are not limited to a specific meaning for a therapeutically effective amount of an agent capable of hindering and/or inhibiting aberrant cartilage formation and/or osteoclast resorption within joints, muscles, tendons, ligaments, connective tissue, and/or bones experiencing or at risk of experiencing ankylosis. In some embodiments, a therapeutically effective amount of an agent capable of hindering and/or inhibiting aberrant cartilage formation and/or osteoclast resorption within joints, muscles, tendons, ligaments, connective tissue, and/or bones experiencing or at risk of experiencing ankylosis is any dosage amount and duration that accomplishes is effective in treating, delaying progression of, or reducing the severity of disorders characterized with aberrant cartilage formation and/or osteoclast resorption within joints, muscles, tendons, ligaments, connective tissue, and/or bones experiencing or at risk of experiencing ankylosis.

In some embodiments, the subject is a mammalian subject (e.g., mouse, horse, human, cat, dog, gorilla, chimpanzee, etc.). In some embodiments, the subject is a human patient suffering from or at risk of suffering from a disorder characterized with aberrant cartilage formation and/or osteoclast resorption within joints, muscles, tendons, ligaments, connective tissue, and/or bones experiencing or at risk of experiencing ankylosis (e.g., ankylosing spondylitis; heterotopic ossification). In some embodiments, the subject is a human subject having a HLA-B27 mutation within the HLA-B gene.

The invention also provides kits comprising one or more capable of hindering and/or inhibiting aberrant cartilage formation and/or osteoclast resorption within joints, muscles, tendons, ligaments, connective tissue, and/or bones experiencing or at risk of experiencing ankylosis and instructions for administering the agent to an animal. The kits may optionally contain one or more other therapeutic agents.

In certain embodiments, the present invention provides methods for treating, delaying progression of, or reducing the severity of spinal cord injuries through administering to a subject (e.g., a human subject having experienced a spinal cord injury) a therapeutic agent capable of inhibiting and/or hindering TGF-β activity at and/or around a site of spinal cord injury.

In some embodiments, such administration results in inhibiting, delaying progression of, or reducing the severity of fibrotic scar formation at and/or around a site of spinal cord injury. In some embodiments, such administration results in inhibiting, delaying progression of, or reducing the severity of fibroblast differentiation of mesenchymal stromal/stem cells (MSCs) at and/or around a site of spinal cord injury.

In certain embodiments, the present invention provides methods for treating, delaying progression of, or reducing the severity of fibrotic scar formation at and/or around a site of spinal cord injury through administering to a subject (e.g., a human subject having experienced a spinal cord injury) a therapeutic agent capable of inhibiting and/or hindering TGF-β activity at and/or around a site of spinal cord injury.

In certain embodiments, the present invention provides methods for treating, delaying progression of, or reducing the severity of fibroblast differentiation of MSCs at and/or around a site of spinal cord injury through administering to a subject (e.g., a human subject having experienced a spinal cord injury) a therapeutic agent capable of inhibiting and/or hindering TGF-β activity at and/or around a site of spinal cord injury.

In some embodiments, the TGF-β inhibitor pertains to a small molecule, antibody or functional portion or fragment thereof, proteins, peptides, siRNAs, antagonists, agonists, compounds, or nucleotide constructs which either reversibly or irreversibly bind TGF-β and prevent its binding to a TGF-β receptor on a cell or tissue in a subject. The term can also mean a small molecule, antibody or functional portion or fragment thereof, proteins, peptides, siRNAs, antagonists, agonists, compounds, or nucleotide constructs which either reversibly or irreversibly bind TGF-β receptors in an antagonistic manner such that TGF-β and its analogs or derivatives cannot stimulate the TGF-β receptors in cells and tissues in a subject.

Examples of TGF-β inhibitors include, for example, antibodies such as (1D11) (see, Wang, et al., J. Clin. Invest. 2018, 128(2):846-860), Fresolimumab, Galunisertib, Lerdelimumab (CAT-152), Metelimumab (CAT-192), GC-1008, SR-2F, and 2G7, small molecule inhibitors such as GW788388 (4-{-[4-3-(Pyridin-2-yl)-1H-pyrazol-4-yl]-pyridin-2-yl}-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxo1-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1 and Lucanix, soluble antagonists such as TGFβRII:Fc, and Betaglycan/TGFβIII. In some embodiments, the TGF-β inhibitor is TGF-β type 1 receptor inhibitor (TbR1I) (see, Qin, et al., Annals. N.Y. Acad. Sci. 1433:29-40).

In any of the described embodiments, the TGF-β inhibitor is formulated to be administered in any desirable manner (e.g., locally, orally, systemically, intravenously, intraarterially, subcutaneously, or intrathecally). The invention also provides pharmaceutical compositions comprising the TGF-β inhibitor in a pharmaceutically acceptable carrier.

Such methods are not limited to a specific meaning for a therapeutically effective amount of TGF-β inhibitor capable of treating, delaying progression of, or reducing the severity of spinal cord injuries. In some embodiments, a therapeutically effective amount is an amount capable of inhibiting, delaying progression of, or reducing the severity of fibrotic scar formation at and/or around a site of spinal cord injury. In some embodiments, a therapeutically effective amount is an amount capable of inhibiting, delaying progression of, or reducing the severity of fibroblast differentiation of mesenchymal stromal/stem cells (MSCs) at and/or around a site of spinal cord injury.

In some embodiments, the subject is a mammalian subject (e.g., mouse, horse, human, cat, dog, gorilla, chimpanzee, etc.). In some embodiments, the subject is a human patient suffering from a spinal cord injury.

The invention also provides kits comprising an agent capable of inhibiting TGF-b activity at and/or around a site of spinal cord injury and instructions for administering the agent to an animal. The kits may optionally contain one or more other therapeutic agents.

### BRIEF DESCRIPTION OF DRAWINGS

FIG. 1. μCT scan of calcified ligaments of AS patients. a. An illustration of ligaments of spine. b. Unaided eye view of limited calcified ligaments of spinous process (Interspinous ligament with Supraspinous ligament and/or Ligamentum flavum) specimen and its μCT scan. c. Unaided eye view of partial calcified ligaments of spinous process (Interspinous ligament with Supraspinous ligament and/or Ligamentum flavum) specimen and its μCT scan. d. Unaided eye view of fully calcified interspinous ligament specimen and its μCT scan. e. Unaided eye view of mature bony ligaments of spinous process (Interspinous ligament with Supraspinous ligament and/or Ligamentum flavum) specimen its μCT scan. f Quantification of heterotopic bone volume in ligaments of spinous process. g. An illustration of ligaments of hip joints. h. Unaided eye view of calcified ligaments of Hip joint specimen and its μCT scan. i. Quantification of heterotopic bone volume in ligaments of Hip joint.

FIG. 2. Elevated TGF-β levels in the early inflammatory stage of AS. a. H&E staining and b. SOFG staining of normal Interspinous ligaments and inflamed Interspinous ligaments. In AS group, the right panels show higher magnification of the boxed area in the left panels Scale bar: 100 μm (right two panels); 25 μm (left panel). c. Immunostaining and d. quantitative analysis of CD68 positive cells (brown) in the Interspinous ligaments (sagittal view) in normal ligaments and inflamed ligaments. The bottom panels show higher magnification of the boxed area in the top panels. Scale bar: 100 μm (top panels); 25 μm (bottom panels). e. Immunostaining and f quantitative analysis of pSmad2/3-positive cells(brown) in the Interspinous ligaments (sagittal view) in normal ligaments and inflammatory ligaments. The bottom panels show higher magnification of the boxed area in the top panels. Scale bar: 100 μm (top panels); 25 μm (bottom panels). g. Immunostaining and h. quantitative analysis of pSmad1/5/8-positive cells(brown) in the Interspinous ligaments (sagittal view) in normal ligaments and inflammatory ligaments. The bottom panels show higher magnification of the boxed area in the top panels. Scale bar: 100 μm (top panels); (bottom panels).

FIG. 3. Chondrocyte differentiation and cartilage formation in the AS interspinous ligaments prior to calcification. a. H&E staining and b. SOFG staining of normal Interspinous ligaments and Chondrogenic Interspinous ligaments. In AS group, the right panels show higher magnification of the boxed area in the left panels. Scale bar: 100 μm (right two panels); (left panel). c. Immunostaining and d. quantitative analysis of Collagen II-positive cells(brown) in the Interspinous ligaments (sagittal view) in normal ligaments and Chondrogenic ligaments. The bottom panels show higher magnification of the boxed area in the top panels. Scale bar: 100 μm (top panel); 25 μm (bottom panel). e. Immunostaining and f quantitative analysis of pSmad2/3-positive cells(brown) in the Interspinous ligaments (sagittal view) in normal ligaments and chondrogenic ligaments. The bottom panels show higher magnification of the boxed area in the top panels. Scale bar: 100 μm (top panel); 25 μm (bottom panel). g. Immunostaining and h. quantitative analysis of pSmad1/5/8-positive cells(brown) in the Interspinous ligaments (sagittal view) in normal ligaments and inflammatory ligaments. The bottom panels show higher magnification of the boxed area in the top panels. Scale bar: 100 μm (top panels); 25 μm (bottom panels).

FIG. 4. Endochondral ossification of HO progression during AS progression in patients. a. H&E staining and b. SOFG staining of normal Interspinous ligaments and endochondral ossified Interspinous ligaments. In AS group, the right panels show higher magnification of the boxed area in the left panels. Scale bar: 100 μm (right two panel); 25 μm (left panel). c. Immunostaining and d. quantitative analysis of Collagen II-positive cells(brown) in the Interspinous ligaments (sagittal view) in normal ligaments and Chondrogenic ligaments. The bottom panels show higher magnification of the boxed area in the top panels. Scale bar: 100 μm(top panels); 25 μm (bottom panels). e. TRAP positive cells (red) and f quantitative analysis of the number of TRAP positive osteoclast(red) surface (OCS) per bone surface (BS). The bottom panels show higher magnification of the boxed area in the top panels. Scale bar: 100 μm (top panel); 25 μm (bottom panel). g. Immunostaining and h. quantitative analysis of the number of CD68-positive osteoclast(brown) surface (OCS) per bone surface (BS). The bottom panels show higher magnification of the boxed area in the top panels. Scale bar: 100 μm (top panel); 25 μm (bottom panel). i. Immunostaining and j. quantitative analysis of pSmad2/3 positive cells(brown) in the Interspinous ligaments (sagittal view) in normal ligaments and endochondral ossified ligaments. The bottom panels show higher magnification of the boxed area in the top panels. Scale bar: 100 μm (top panel); 25 μm (bottom panel). k. Immunostaining and l. quantitative analysis of OSX positive cells(brown) in the Interspinous ligaments (sagittal view) in normal ligaments and endochondral ossified ligaments. The bottom panels show higher magnification of the boxed area in the top panels. Scale bar: 100 μm (top panel); 25 μm (bottom panel). m. Immunostaining and n. quantitative analysis of pSmad1/5/8-positive cells(brown) in the Interspinous ligaments (sagittal view) in normal ligaments and inflammatory ligaments. The bottom panels show higher magnification of the boxed area in the top panels. Scale bar: 100 μm (top panels); 25 μm (bottom panels).

FIG. 5. Neonatal mice completely recovered from SCI. (A) Timeline of different time points. (B) Representative images of immunofluorescent analysis of pSmad2 (red), PDGFR-β (green), and DAPI (blue) in the spinal cord lesion site in neonatal mice on days 2, 7, and 12 with or without SCI. Scale bars=200 μm. Right images are high-resolution versions of the boxed regions in the left images. Scale bars=50 μm. (C, D) Quantitative analysis of the number of pSmad2⁺ cells and PDGFR-β⁺ cells (*P<0.05, ** P<0.01, ****P<0.0001, n=6). (E) Representative images of immunofluorescent analysis of β-III-tubulin (green), collagen III (red), and DAPI (blue) in the spinal cord lesion site in neonatal mice on days 2 and 12, and adult mice with or without SCI. Scale bars=200 μm. (F, G) Quantitative analysis of the intensity mean value of collagen III⁺ fibrotic scar and β-III-tubulin⁺ nerves (*P<0.05, **P<0.01, ***P<0.001, ****P<0.0001, n=6). (H) Schematic pattern for the whole hypothesis. Statistical significance was determined by multifactorial ANOVA, and all data are shown as means±standard deviations.

FIG. 6. Functional recovery in Tgfbr2_{Glast-creER}^{-/-}mice. (A) Quantitative analysis of hindpaw withdrawal time responding to temperature (hot plate test, *P<0.05, n=8). (B, C) Quantitative analysis of hindpaw withdrawal frequency responding to mechanical stimulation (von Frey test, 0.7 mN and 3.9 mN, *P<0.05, **P<0.01, n=8). (D) Quantitative analysis of BMS score between Tgfbr2^{flox/flox} control mice after SCI, Tgfbr2_{Glast-creER}^{-/-} mice after SCI, and Tgfbr2^{flox/flox} control mice without SCI (n=8). (E-H) Representative footprints of animal walking 8 weeks after SCI and quantitative analysis of CatWalk, including print length, stride length, and print area (*P<0.05, n=8). PWF: paw withdrawal frequency. LF: left forepaw, RF: right forepaw, RH: right hindpaw. Statistical significance was determined by multifactorial ANOVA, and all data are shown as means ±standard deviations.

FIG. 7. LepR⁺ MSCs were the primary cells for fibrotic scar formation. (A, B) Genetic strategy to tracing type A pericytes and the timeline of tamoxifen injection. (C) Representative images of immunofluorescent analysis of tdT⁺ (red) type A pericytes, PDGFR-β⁺ (green) pericytes, and DAPI (blue) staining of nuclei in the spinal cord lesion site of T10 wild-type (WT) mice at 2 weeks after SCI. Scale bars=200 μm. Right images are high-resolution versions of the boxed regions in the left images. Scale bars=50 μm. (D) Representative images of immunofluorescent analysis of collagen III⁺ (green) fibrotic scar, tdT⁺ (red) type A pericytes, and DAPI (blue) staining of nuclei in spinal cord lesion site of T10 in 13C4 group control mice and 1D11 group mice at 4 weeks after SCI. Scale bars=200 μm. (E) Quantitative analysis of % of PDGFR-β cells/tdTomato⁺ type A pericytes field (****P<0.0001, n=4). (F) Quantitative analysis of percentage of collagen III⁺/tdTomato⁺ type A pericytes field (***P<0.001, n=4). (G) Representative images of immunofluorescent analysis of LepR⁺ (green) MSCs, tdT⁺ (red) type A pericytes, and DAPI (blue) staining of nuclei in sham group mice, 13C4 group control mice, and 1D11 group mice at 4 weeks after SCI. (H) Quantitative analysis of the percentage of LepR⁺ cells/tdTomato⁺ cells (**P<0.01, n=4). (I) Representative images of immunofluorescent analysis of LepR⁺ (green) MSCs, collagen III⁺ (green) fibrotic scar, and DAPI (blue) staining of nuclei. Scale bars=200 μm. (J) Quantitative analysis of the percentage of collagen III⁺/LepR⁺ MSCs field (***P<0.001, n=4). (K) Representative images of immunofluorescent analysis of LepR⁺ (green) MSCs, FSP1⁺ (red) type A pericytes, and DAPI (blue) staining of nuclei. Scale bars=200 μm. (L) Quantitative analysis of the percentage of LepR⁺ MSCs/FSP1⁺ cells field (***P<0.001, n=4). Statistical significance was determined by multifactorial ANOVA, and all data are shown as means±standard deviations.

FIG. 8. Systemic injection of TGF-β-neutralizing antibody promoted neurological function recovery after SCI. (A) Timeline and concentration of TGF-β-neutralizing antibody 1D11 injection. (B) Representative images of immunofluorescent analysis of PGP9.5⁺ (red) nerve fibers, fibronectin⁺ (green) fibrotic scar, and DAPI (blue) staining of nuclei at 4 weeks after SCI. Scale bars=500 μm. (C) Representative images of immunofluorescent analysis of nerve axon-specific β-III-tubulin⁺ (green), collagen III⁺ (red) fibrotic scar, and DAPI (blue) staining of nuclei at 4 weeks after SCI. Scale bars=200 μm. Right images are high-resolution versions of the boxed regions in the left images. Scale bars=50 μm. (D, E) Quantitative analysis of the intensity value of PGP9.5⁻ and fibronectin (**P<0.01, n=6). (F) Quantitative analysis of the PGP9.5⁺ nerve gap. (G) Quantitative analysis of the intensity value of collagen III (**P<0.01, n=6). (H) Representative images of immunofluorescent analysis of neurotransmitter marker 5-HT⁺ (red), nerve axon-specific-β-III-tubulin⁺ (green), and DAPI (blue) staining of nuclei in the spinal cord lesion site of T10 in sham group mice, 13C4 group control mice, and 1D11 group mice at 4 weeks after SCI. Scale bars=200 μm. Right images are high-resolution versions of the boxed regions in the left images. Scale bars=50 μm. (I, J) Quantitative analysis of the intensity value of 5-HT and β-III-tubulin (*P<0.05, **P<0.01, n=6). (K-N) ELISA analysis showing the concentration of TGF-β in spinal cord and serum after SCI between 13C4 group control mice, 1D11 group mice, and 13C4 group control mice without surgery (sham) (*P<0.05, **P<0.01, n=4). (O) Representative images of immunofluorescent analysis of pSmad2⁺ (red), PDGFR-β⁺ (green) pericytes, and DAPI (blue) staining of nuclei at 7 days after SCI. Scale bars=10 μm. (P, Q) Quantitative analysis of the intensity mean value of PDGFR-β and the number of pSmad2⁺ cells (*P<0.05, **P<0.01, n=6). (R) Representative Western blots showing the activation of pSmad signaling. (S) Quantitative analysis of the pSmad2/β-actin ratio in 13C4 group control mice after SCI, 1D11 group mice after SCI, and 13C4 group control mice without surgery (sham) (**P<0.01, ***P<0.001, ****P<0.0001, n=3). Statistical significance was determined by multifactorial ANOVA, and all data are shown as means±standard deviations.

FIG. 9. Glial fibrillary acidic protein (GFAP), collagen III, and fibronectin immunostaining of mouse SCI with approaches to inhibit TGF-β activity. (A) Representative images of immunofluorescent analysis of collagen III⁺ (red) fibrotic scar, GFAP⁺ (green) glial scar, and DAPI (blue) staining of nuclei in the spinal cord lesion site of T10 in Tgfb1^{flox/flox} control mice and Tgfb1_{LysM-cre}^{-/-} mice at 4 weeks after SCI. Scale bars=200 μm. (B, C) Quantitative analysis of the intensity values of collagen III and GFAP (****P<0.0001, n=6). (D) Representative images of immunofluorescent analysis of fibronectin⁺ (red) fibrotic scar, GFAP⁺ (green) glial scar and DAPI (blue) staining of nuclei in 13C4 group control mice and 1D11 group mice at 4 weeks after SCI. Scale bars=200 μm. (E) Quantitative analysis of the intensity value of fibronectin and GFAP (****P<0.0001, n=6). Statistical significance was determined by multifactorial ANOVA, and all data are shown as means±standard deviations.

FIG. 10. Functional recovery after global knockout of TGF-β. (A) Quantitative analysis of hindpaw withdrawal time responding to temperature (hot plate test, *P<0.05, **P<0.01, n=8). (B, C) Quantitative analysis of hindpaw withdrawal frequency responding to mechanical stimulation (von Frey test, 0.7 mN and 3.9 mN, *P<0.05, **P<0.01, ****P<0.0001, n=8). (D) Quantitative analysis of BMS score between 13C4 control mice after SCI, 1D11 mice after SCI, and 13C4 control mice without SCI. (E-H) Quantitative analysis of CatWalk, including print length, stride length, and print area (*P<0.05, n=8). PWF: paw withdrawal frequency. LF: left forepaw, RF: right forepaw, RH: right hindpaw. Statistical significance was determined by multifactorial ANOVA, and all data are shown as means±standard deviations.

FIG. 11. TGF-β-neutralizing antibody 1D11 more effectively inhibited fibrotic scar formation and promoted axonal growth relative to the 3 other knockout mice. (A, B) Representative images of immunofluorescent analysis of neurotransmitter marker 5-HT⁺ (red) downstream distance and fibrotic scar area between different knockout mice. (C, D) Quantitative analysis of the downstream distance of 5-HT⁺ neurotransmitters (mm) and fibrotic scar area (mm²) (**P<0.01, ***P<0.001, ****P<0.0001, ****P<0.0001, n>4). Statistical significance was determined by multifactorial ANOVA, and all data are shown as means±standard deviations.

FIG. 12. Single-cell sequencing revealed elevated active TGF-β induced fibroblast differentiation of MSCs after SCI. (A) Clustered and annotated data from all sample groups (1D11, 13C4, and Sham) were plotted on UMAP coordinates. The following cell types were identified in our data: endothelial cells, fibroblasts, micro and macroglia, mesenchymal stem cells, neurons, and pericytes. (B) UMAP plot of cell type annotations split by sample group. (C) Heat map of enriched expression of TGF-β pathway genes in pericytes from the 1D11 versus 13C4 sample groups. Pericytes in the 13C4 group exhibited significant enrichment in expression of TGF-β pathway genes relative to the 1D11 group. (D-G) Violin plots of TGF-β ligand and receptor expression across cell types, split by sample group. (H) Dot plot of the mean expression and fraction of cells in a given sample group expressing three key axon growth regulators: two growth inhibitors (Sema3a and Ephb2) and one growth factor (Cd248). (I) Dot plot of significant (p<0.05) ligand-receptor interactions between cell types of interest, as determined by CellPhoneDB. Several significant ligand-receptor interactions were observed amongst genes from pro-fibrotic and TGF-β signaling pathways.

FIG. 13. Results of unbiased classification of single cell data and downstream differential expression and enrichment analyses. (A) Cells are plotted on UMAP coordinates and colored by SingleCellNet (SCN) annotation. SCN was trained on Tabula Muris Senis skeletal muscle training data and each query cell was assigned the class label for which SCN returned the highest classification score. (B) Heat map of top 20 differentially expressed genes per cell type, as computed in Seurat using FindAllMarkers and the wilcox rank sum test. (C) Dot plot of the top 10 differentially expressed genes per cell type.

FIG. 14. Trajectory inference of developmentally related cell types reveals two putative lineages to generate fibroblasts through an MSC intermediate. (A) Heat map of dynamically expressed genes across pseudotime for the lineage representing the transition of endothelial cells to fibroblasts through an MSC intermediate. (B-C) Pseudotime curves plotted on harmonized principal components that illustrate two lineages: EC→MSC→fibroblast and pericyte→MSC→fibroblast. (D-E) Pseudotime values for the first and second lineages, respectively.

FIG. 15. Elimination of macrophage lineage cells reduced TGF-β activity in SCI mice. (A) Timeline of diphtheria toxin (DT) injection and sample collection. (B) Schematic diagram of the location of SCI. (C) Representative images of immunofluorescent analysis of PGP9.5⁺ (red) nerve fibers, fibronectin⁺ (green) fibrotic scar, and DAPI (blue) staining of nuclei at 4 weeks after SCI. Scale bars=200 μm. (D-F) Quantitative analysis of the intensity values of PGP9.5 and fibronectin (*P<0.05, n=6). Quantitative analysis of the PGP9.5⁺ nerve gap (**P<0.01, n=6). (G-J) ELISA analysis showing the concentration of TGF-β in spinal cord and serum after SCI (*P<0.05, **P<0.01, n=4). (K) Representative Western blots showing the activation of pSmad signaling. (L) Quantitative analysis of the pSmad2/β-actin ratio in iDTR_{LysM-cre}^{-/-} knockout mice after SCI, iDTR^{flox/flox} control mice after SCI, and iDTR^{flox/flox} control mice without surgery (sham) (*P<0.05, ***P<0.001, n=3). (M) Representative images of immunohistochemical analysis of pSmad2 (brown) at 3, 5, and 7 days and 2 and 4 weeks after SCI. Scale bars=50 μm. (N) Quantitative analysis of the number of pSmad2⁺ cells at different time points (**P<0.01, n=6). Statistical significance was determined by multifactorial ANOVA, and all data are shown as means±standard deviations.

FIG. 16. Efficient inhibition of TGF-β activity in iDTR_{LysM-cre}^{-/-} mice. (A, C) Representative images of immunohistochemical analysis of F4/80⁺ (brown) macrophage in spleen and spinal cord at 1, 7, 14, 28, and 56 days after SCI. Scale bars=50 μm. (B, D) Quantitative analysis of F4/80⁺ cells at different time points (***P<0.001, n=6). Statistical significance was determined by multifactorial ANOVA, and all data are shown as means±standard deviations.

FIG. 17. Experimental design of treatment timelines and SCI procedure. (A) Representative images showing the surgical procedures. (B) iDTR_{LysM-cre}^{-/-} mice were treated with 100 ng/0.2 mL of diphtheria toxin (DT) to knock out macrophages for 3 days before SCI and 3 times a week after surgery until the mice were humanely killed at 1, 3, 5, 7, 14, 28, or 56 days. Mice were treated with 5 mg/kg body weight of the TGFβ-neutralizing antibody 1D11 3 times a week until the mice were humanely killed. TdTomato_{Glast-cre}^{-/-} mice were treated with tamoxifen for 2 mg of 20 mg/mL per mouse for 5 consecutive days. Vehicle mice received the same number of injections of the solvent without DT, 1D11, and tamoxifen.

FIG. 18. Conditional knockout of transforming growth factor-β 1 (Tgfβ1) in macrophage lineage cells reduced fibrotic scar formation in adult spinal cord injury (SCI) mice. (A) Representative images of immunofluorescent analysis of collagen III⁺ fibrotic scar at 1, 2, 4, and 8 weeks in iDTR_{LysM-cre}^{-/-} knockout mice after SCI, iDTR^{flox/flox} control mice after SCI, and iDTR^{flox/flox} control mice without surgery (sham). Scale bars=200 μm. (B) Quantitative analysis of collagen III⁺ area at different time points (*P<0.05, **P<0.01, ***P<0.001, n=6). (C) Schematic diagram of TGF-β1 knockout in LysM-cre lineage cells. (D) Representative images of immunofluorescent analysis of protein gene product 9.5⁺ (PGP9.5⁺) (red) nerve fibers, fibronectin⁺ (green) fibrotic scar, and 4′,6-diamidino-2-phenylindole (DAPI) (blue) staining of nuclei at 4 weeks after SCI. Scale bars=200 μm. (E, F) Quantitative analysis of the intensity value of PGP9.5 and fibronectin (*P<0.05, **P<0.01, n=6). (G) Quantitative analysis of the PGP9.5⁺ nerve gap. (H) Representative images of immunofluorescent analysis of nerve axon-specific-62 -III-tubulin⁺ (green), collagen III⁺ (red) fibrotic scar, and DAPI (blue) staining of nuclei at 4 weeks after SCI. Scale bars=200 μm. (I, J) Quantitative analysis of the intensity value of collagen III and β-III-tubulin⁺ (*P<0.05, **P<0.01, n=6). (K) Representative images of immunofluorescent analysis of neurotransmitter marker 5-HT⁺ (red), nerve axon-specific β-III-tubulin⁺ (green), and DAPI (blue) staining of nuclei in spinal cord lesion site of T10 in Tgfb1^{flox/flox} sham group mice, Tgfb1^{flox/flox} control mice, and Tgfb1_{LysM-cre}^{-/-}mice at 4 weeks after SCI. Scale bars=200 μm. Right images are high resolution versions of the boxed regions in the left images, scale bars=50 μm. (L) Quantitative analysis of the intensity value of 5-HT (*P<0.05, n=6). (M-P) ELISA analysis showing the concentration of TGF-β in spinal cord and serum after SCI (*P<0.05, **P<0.01, n=4). (Q) Representative Western blots showing the activation of phosphorylated Smad (pSmad) signaling. (R) Quantitative analysis of the pSmad2/β-actin ratio in Tgfb1^{flox/flox} control mice after SCI, Tgfb1_{LysM-cre}^{-/-} mice after SCI, and Tgfb1^{flox/flox} control mice without surgery (sham) (*P<0.05, **P<0.01, ***P<0.001, n=3). (S) Representative images of immunofluorescent staining of pSmad2⁺ (red), platelet-derived growth factor receptor-β⁺ (PDGFR-β⁺) (green) pericytes, and DAPI (blue) staining of nuclei at 7 days after SCI. Scale bars=10 μm. (T) Representative immunofluorescence images of PDGFR-β⁺ (green) pericytes and DAPI (blue) staining of nuclei at 2 weeks after SCI. Scale bars=200 μm. (U) Quantitative analysis of the number of pSmad2⁺ cells (**P<0.01, n=6). (V) Quantitative analysis of the number of PDGFR-β⁺ cells (**P<0.01, n=6). Statistical significance was determined by multifactorial analysis of variance (ANOVA), and all data are shown as means±SD.

FIG. 19. Elimination of macrophage lineage cells promoted functional recovery in SCI mice. (A) Representative images of immunofluorescent analysis of neurotransmitter marker 5-HT⁺ (red), nerve axon-specific β-III-tubulin⁺ (green), and DAPI (blue) staining of nuclei in the spinal cord lesion site of T10 in iDTR_{LysM-cre}^{-/-} knockout mice and iDTR^{flox/flox} control mice at 4 weeks after SCI. Scale bars=200 μm. Right images are high-resolution versions of the boxed regions in the left images. Scale bars=50 μm. (B, C) Quantitative analysis of the intensity values of 5-HT and β-III-tubulin (*P<0.05, n=6). (D, E) Schematic diagram of hot plate test and von Frey test. (F) Quantitative analysis of hindpaw withdrawal time responding to temperature (hot plate test, *P<0.05, **P<0.01, n=8). (G, H) Quantitative analysis of hindpaw withdrawal frequency responding to mechanical stimulation (von Frey test, 0.07 mN and 0.4 mN, **P<0.01, ****P<0.0001, n=8). (I) Quantitative analysis of BMS score between iDTR^{flox/flox} control mice after SCI, iDTR_{LysM-cre}^{-/-} knockout mice after SCI, and iDTR^{flox/flox} control mice without SCI (*P<0.05, **P<0.01, n=8). (J-M) Representative footprints of animal walking 8 weeks after SCI and quantitative analysis of CatWalk, including print length, stride length, and print area (*P<0.05, **P<0.01, n=8). PWF: paw withdrawal frequency. LF: left forepaw, RF: right forepaw, RH: right hindpaw. Statistical significance was determined by multifactorial ANOVA, and all data are shown as means±standard deviations.

FIG. 20. Conditional knockout of TgfB1 in macrophage lineage cells promoted functional recovery in SCI mice. (A, B) Schematic patterns of hot plate test and von frey test. (A, C) Quantitative analysis of hindpaw withdrawal time responding to temperature (hot plate test, **P<0.01, n=8). (B, D, E) Quantitative analysis of hindpaw withdrawal frequency responding to mechanical stimulation (von Frey test, 0.7 mN and 3.9 mN, *P<0.05, **P<0.01, n=8). (F) Quantitative analysis of Basso Mouse Scale (BMS) score between Tgfb1^{flox/flox} control mice after SCI, Tgfb1_{LysM-cre}^{-/-} mice after SCI, and Tgfb1^{flox/flox} control mice without SCI (*P<0.05, n=8). (G-J) Representative footprints of animal walking 8 weeks after SCI and quantitative analysis of CatWalk, including print length, stride length, and print area (*P<0.05, **P<0.01, n=8). PWF: paw withdrawal frequency. LF: left forepaw, RF: right forepaw, RH: right hindpaw. Statistical significance was determined by multifactorial ANOVA, and all data are shown as means±standard deviations.

FIG. 21. Conditional knockout of TGF-β type 2 receptor (Tgfbr2) in pericytes reduced fibrotic scar formation in SCI mice. (A) Schematic diagram of Tgfbr2 knockout in Glast-Cre⁺ pericytes. (B) Representative images of immunofluorescent analysis of PGP9.5⁺ (red) nerve fibers, fibronectin⁺ (green) fibrotic scar, and DAPI (blue) staining of nuclei at 4 weeks after SCI. Scale bars=200 μm. (C, D) Quantitative analysis of the intensity value of PGP9.5 and fibronectin (***P<0.001, n=6). (E) Quantitative analysis of the PGP9.5⁺ nerve gap. (F) Representative images of immunofluorescent analysis of nerve axon—specific β-III-tubulin⁺ (green), collagen III⁺ (red) fibrotic scar, and DAPI (blue) staining of nuclei at 4 weeks after SCI. Scale bars=200 μm. Right images are high resolution versions of the boxed regions in the left images. Scale bars=50 μm. (G, H) Quantitative analysis of the intensity value of collagen III and β-III-tubulin (*P<0.05, n=6). (I) Representative images of immunofluorescent analysis of neurotransmitter marker 5-HT⁺ (red), nerve axon-specific β-III-tubulin⁺ (green), and DAPI (blue) staining of nuclei in spinal cord lesion site of T10 in Tgfbr2^{flox/flox} sham group mice, Tgfbr2^{flox/flox} control mice, and Tgfbr2_{Glast-creER}^{-/-} mice at 4 weeks after SCI. Scale bars=200 μm. Right images are high-resolution versions of the boxed regions in the left images. Scale bars=50 μm. (J) Quantitative analysis of the intensity value of 5-HT (*P<0.05, n=6). (K) Representative images of immunofluorescent analysis of pSmad2⁺ (red), PDGFR-β⁺ (green) pericytes and DAPI (blue) staining of nuclei at 7 days after SCI. Scale bars=10 μm. (L, M) Quantitative analysis of the intensity mean value of PDGFR-β and the number of pSmad2⁺ cells (*P<0.05, n=6). (N) Representative Western blots showing the activation of pSmad signaling. (O) Quantitative analysis of the pSmad2/β-actin ratio in Tgfbr2^{flox/flox} control mice after SCI, Tgfbr2_{Glast-creER}^{-/-} mice after SCI, and Tgfbr2^{flox/flox} control mice without surgery (sham) (**P<0.01, ***P<0.001, ****/ P<0.0001, n=3). Statistical significance was determined by multifactorial ANOVA, and all data are shown as means±standard deviations.

### DETAILED DESCRIPTION OF THE INVENTION

Ankylosing spondylitis (AS) is a chronic inflammatory arthritis with progressive fusion of axial joints. While anti-inflammatory treatment such as anti-TNF-α antibody therapy effectively suppresses inflammation, it does not effectively halt the progression of ankylosing to bring disease remission.

Experiments conducted during the course of developing embodiments for the present invention determined that the autoimmune inflammation of highly familial and heritable AS generates a microenvironment that promotes chondrogenesis in the spine ligaments. H&E staining of AS ligament demonstrated chondrocyte differentiation in the ligaments of early stage AS, and Safranin O and fast green staining showed cartilage formation in the ligaments of early stage AS, which was subsequently calcified. Moreover, large number of giant osteoclasts were found at bony surface and their bone resorption activity generated marrow with high levels of active TGF-β. As a result, the high levels of active TGF-β recruited large number of Osterix⁺ osteoprogenitors for the new bone formation. Particularly, Osterix⁺ osteoprogenitors were found not in the osteoclast resorption areas, indicating uncoupled bone resorption and formation. Even at late and mature stage, the uncoupled osteoclast resorption bony interspinous ligament activates TGF-β to drive progression of ossification in AS patients. The osteoclast resorption of calcified cartilage-initiated ossification in progression of AS is a similar pathological process pathogenesis of acquired heterotopic ossification. The finding of cartilage formation in the ligaments of AS uncovered the pathogenesis of spine fusion and explained why anti-inflammatory treatments do not slow the rate of ankylosing from new bone formation in the spine soft tissue. Thus, such results indicate that inhibition of cartilage formation, osteoclast resorption and/or TGF-β activity within at risk ligaments as an effective therapy for treating, prevention and/or ameliorating the symptoms of AS.

Spinal cord injury (SCI) often causes disability in humans and other mammals. Experiments conducted during the course of developing embodiments for the present invention determined that fibrotic scar formation at injury sites prevents recovery after SCI and that the inhibition of fibrotic scar formation significantly improved recovery in adult mice. Neonatal mice were able to fully recover from SCI because they do not experience fibrotic scar formation. Active transforming growth factor-β1 (TGF-β1) was significantly elevated at SCI sites to recruit mesenchymal stromal/stem cells (MSCs) and induce fibroblast differentiation. Eliminating macrophage lineage cells in LysM-cre::iDTR^{flox/flox} mice significantly decreased TGF-β activity, suggesting macrophages as primary source of TGF-β1. Moreover, TGF-β activity was significantly decreased after selective deletion of TGF-β1 in macrophages in LysM-cre: Tgfb1^{flox/flox} mice. Knocking out of TGF-β type 2 receptor in pericytes of Glast-creERT2::Tgfbr2^{flox/flox} mice also decreased fibrotic scar formation. Interestingly, TGF-β-neutralizing antibody 1D11 treatment induced a greater inhibition of scar formation than any of the knockout mice. Single-cell RNA sequencing revealed that TGF-β downstream signaling was significantly enriched in MSCs, fibroblasts, pericytes, and endothelial cells after SCI, and the lineage trajectory indicates that pericytes differentiate to fibroblasts via MSCs. Most importantly, neonatal mice did not have active TGF-β at the injury site and they recovered completely after SCI. Thus, fibrotic scar may be the primary obstacle that prevents recovery after SCI.

Accordingly, the present invention provides compositions and methods directed to treating, delaying progression of, or ameliorating symptoms related to disorders characterized with TGF-b activity (e.g., disorders characterized with aberrant cartilage formation and/or osteoclast resorption (e.g., ankylosing spondylitis)) (e.g., spinal cord injuries) (e.g., fibrotic scar formation following spinal cord injury) through inhibition of TGF-b activity (e.g., thereby hindering and/or inhibiting aberrant cartilage formation and/or osteoclast resorption within joints, muscles, tendons, ligaments, connective tissue, and/or bones experiencing or at risk of experiencing ankylosis) (e.g., thereby inhibiting fibrotic scar formation following spinal cord injury).

In certain embodiments, the present invention provides methods for treating, delaying progression of, or reducing the severity of disorders characterized with aberrant cartilage formation and/or osteoclast resorption (e.g., ankylosing spondylitis) through hindering and/or inhibiting aberrant cartilage formation and/or osteoclast resorption within joints, muscles, tendons, ligaments, connective tissue, and/or bones experiencing or at risk of experiencing ankylosis. Such methods are not limited to particular type or manner of treating, delaying progression of, or reducing the severity of disorders characterized with aberrant cartilage formation and/or osteoclast resorption (e.g., ankylosing spondylitis) through hindering and/or inhibiting aberrant cartilage formation and/or osteoclast resorption within joints, muscles, tendons, ligaments, connective tissue, and/or bones experiencing or at risk of experiencing ankylosis

In some embodiments, the present invention provides methods for treating, delaying progression of, or reducing the severity of disorders characterized with aberrant cartilage formation and/or osteoclast resorption (e.g., ankylosing spondylitis) through hindering and/or inhibiting aberrant cartilage formation and/or osteoclast resorption within joints, muscles, tendons, ligaments, connective tissue, and/or bones experiencing or at risk of experiencing ankylosis comprising administering to a subject in need thereof a therapeutically effective amount of an agent capable of hindering and/or inhibiting cartilage formation and/or osteoclast resorption within joints, muscles, tendons, ligaments, connective tissue, and/or bones experiencing or at risk of experiencing ankylosis. In some embodiments, such administration results in one or more of the following within joints, muscles, tendons, ligaments, connective tissue, and/or bones experiencing or at risk of experiencing ankylosis: inhibition of cartilage formation; inhibition of osteoclast resorption activity; reduction and/or inhibition of TGF-β expression and/or activity; reduction and/or inhibition of pSmad1/5/8 signaling; reduction and/or inhibition of pSmad2/3 signaling; inhibition and/or hindering of chondrogenesis; inhibition and/or hindering of chondrocyte differentiation; prevention ankylosing spondylitis development; and reduction in the severity of ankylosing spondylitis.

Such methods are not limited to particular types or kinds of disorders characterized with aberrant cartilage formation and/or osteoclast resorption. In some embodiments, the disorder characterized with aberrant cartilage formation and/or osteoclast resorption is ankylosing spondylitis (AS). In some embodiments, the disorder characterized with aberrant cartilage formation and/or osteoclast resorption is heterotopic ossification. In some embodiments, the disorder characterized with aberrant cartilage formation and/or osteoclast resorption is rheumatoid arthritis, metastatic bone disease and/or Paget disease.

Such embodiments are not limited to a particular type of agent capable hindering and/or inhibiting aberrant cartilage formation and/or osteoclast resorption within joints, muscles, tendons, ligaments, connective tissue, and/or bones experiencing or at risk of experiencing ankylosis (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In some embodiments, the agent is capable of one or more of the following within joints, muscles, tendons, ligaments, connective tissue, and/or bones experiencing or at risk of experiencing ankylosis: inhibition of cartilage formation; inhibition of osteoclast resorption activity; reduction and/or inhibition of TGF-β expression and/or activity; reduction and/or inhibition of pSmad1/5/8 signaling; reduction and/or inhibition of pSmad2/3 signaling; inhibition and/or hindering of chondrogenesis; inhibition and/or hindering of chondrocyte differentiation; prevention ankylosing spondylitis development; and reduction in the severity of ankylosing spondylitis.

Examples of TGF-β inhibitors include, for example, antibodies such as (1D11) (see, Wang, et al., J. Clin. Invest. 2018, 128(2):846-860), Fresolimumab, Galunisertib, Lerdelimumab (CAT-152), Metelimumab (CAT-192), GC-1008, SR-2F, and 2G7, small molecule inhibitors such as GW788388 (4-{4-[3-(Pyridin-2-yl)-1H-pyrazol-4-yl]-pyridin-2-yl}-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxo1-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1 and Lucanix, soluble antagonists such as TGFβRII:Fc, and Betaglycan/TGFβRIII. In some embodiments, the TGF-β inhibitor is TGF-β type 1 receptor inhibitor (TβR1I) (see, Qin, et al., Annals. N.Y. Acad. Sci. 1433:29-40).

In some embodiments, the agent is a retinoic acid agonist (e.g., a retinoic acid a agonist; a retinoic acid γ agonist) (e.g., see, Shimono, et al., J. Orthop. Res. 28:217-277, 2010; Pavey, et al., Bone 90 (2016), 159-167; Uchibe, et al., J. Orthop. Res. 35:1096-1105, 2017; Shimono, et al., Nat. Med. 2011 Apr. 17(4): 454-460); WO2016144976A9). RAR agonists may include any agent that activates RAR or sustains retinoic acid so that its activity at RAR increases. This includes both substances that initiate a physiological response when combined with a receptor, as well as substances that prevent the catabolism (or breakdown) of retinoids (for example, retinoic acid), allowing the signal from retinoic acid itself to increase. In some embodiments, the retinoic acid a agonist is selected from ATRA, AM580, AM80 (tamibarotene), BMS753, BD4, AC-93253, AR7, and NRX195183. In some embodiments, the retinoic acid y agonist is selected from Palovartene, CD2665, MM11253, 7a, NRX204647.

In some embodiments, the agent is an angiotensin II receptor antagonist (e.g., losartan, valsartan). An angiotensin II receptor antagonist is an angiotensin receptor blocker that is known to suppress the TGF-β signaling cascade.

In any of the described embodiments, the agent is formulated to be administered in any desirable manner (e.g., locally, orally, systemically, intravenously, intraarterially, subcutaneously, or intrathecally).

In certain embodiments of the invention, combination treatment with the agent capable of hindering and/or inhibiting aberrant cartilage formation and/or osteoclast resorption within joints, muscles, tendons, ligaments, connective tissue, and/or bones experiencing or at risk of experiencing ankylosis and a course of a drug known for treating disorders characterized with aberrant cartilage formation and/or osteoclast resorption (e.g., a drug known for treating treating ankylosing spondylitis).

The invention also provides pharmaceutical compositions comprising the agent capable of hindering and/or inhibiting aberrant cartilage formation and/or osteoclast resorption within joints, muscles, tendons, ligaments, connective tissue, and/or bones experiencing or at risk of experiencing ankylosis in a pharmaceutically acceptable carrier.

Such methods are not limited to a specific meaning for a therapeutically effective amount of an agent capable of hindering and/or inhibiting aberrant cartilage formation and/or osteoclast resorption within joints, muscles, tendons, ligaments, connective tissue, and/or bones experiencing or at risk of experiencing ankylosis. In some embodiments, a therapeutically effective amount of an agent capable of hindering and/or inhibiting aberrant cartilage formation and/or osteoclast resorption within joints, muscles, tendons, ligaments, connective tissue, and/or bones experiencing or at risk of experiencing ankylosis is any dosage amount and duration that accomplishes is effective in treating, delaying progression of, or reducing the severity of disorders characterized with aberrant cartilage formation and/or osteoclast resorption within joints, muscles, tendons, ligaments, connective tissue, and/or bones experiencing or at risk of experiencing ankylosis.

In some embodiments, the subject is a mammalian subject (e.g., mouse, horse, human, cat, dog, gorilla, chimpanzee, etc.). In some embodiments, the subject is a human patient suffering from or at risk of suffering from a disorder characterized with aberrant cartilage formation and/or osteoclast resorption within joints, muscles, tendons, ligaments, connective tissue, and/or bones experiencing or at risk of experiencing ankylosis (e.g., ankylosing spondylitis; heterotopic ossification). In some embodiments, the subject is a human subject having a HLA-B27 mutation within the HLA-B gene.

In certain embodiments, the present invention provides methods for treating, delaying progression of, or reducing the severity of spinal cord injuries through administering to a subject (e.g., a human subject having experienced a spinal cord injury) a therapeutic agent capable of inhibiting and/or hindering TGF-β activity at and/or around a site of spinal cord injury.

In some embodiments, such administration results in inhibiting, delaying progression of, or reducing the severity of fibrotic scar formation at and/or around a site of spinal cord injury. In some embodiments, such administration results in inhibiting, delaying progression of, or reducing the severity of fibroblast differentiation of mesenchymal stromal/stem cells (MSCs) at and/or around a site of spinal cord injury.

In certain embodiments, the present invention provides methods for treating, delaying progression of, or reducing the severity of fibrotic scar formation at and/or around a site of spinal cord injury through administering to a subject (e.g., a human subject having experienced a spinal cord injury) a therapeutic agent capable of inhibiting and/or hindering TGF-β activity at and/or around a site of spinal cord injury.

In certain embodiments, the present invention provides methods for treating, delaying progression of, or reducing the severity of fibroblast differentiation of MSCs at and/or around a site of spinal cord injury through administering to a subject (e.g., a human subject having experienced a spinal cord injury) a therapeutic agent capable of inhibiting and/or hindering TGF-β activity at and/or around a site of spinal cord injury.

In some embodiments, the TGF-β inhibitor pertains to a small molecule, antibody or functional portion or fragment thereof, proteins, peptides, siRNAs, antagonists, agonists, compounds, or nucleotide constructs which either reversibly or irreversibly bind TGF-β and prevent its binding to a TGF-β receptor on a cell or tissue in a subject. The term can also mean a small molecule, antibody or functional portion or fragment thereof, proteins, peptides, siRNAs, antagonists, agonists, compounds, or nucleotide constructs which either reversibly or irreversibly bind TGF-β receptors in an antagonistic manner such that TGF-β and its analogs or derivatives cannot stimulate the TGF-β receptors in cells and tissues in a subject.

Examples of TGF-β inhibitors include, for example, antibodies such as (1D11) (see, Wang, et al., J. Clin. Invest. 2018, 128(2):846-860), Fresolimumab, Galunisertib, Lerdelimumab (CAT-152), Metelimumab (CAT-192), GC-1008, SR-2F, and 2G7, small molecule inhibitors such as GW788388 (4-{4-[3-(Pyridin-2-yl)-1H-pyrazol-4-yl]-pyridin-2-yl}-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxo1-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1 and Lucanix, soluble antagonists such as TGFβRII:Fc, and Betaglycan/TGFβRIII. In some embodiments, the TGF-β inhibitor is TGF-β type 1 receptor inhibitor (TβR1I) (see, Qin, et al., Annals. N.Y. Acad. Sci. 1433:29-40).

In any of the described embodiments, the TGF-β inhibitor is formulated to be administered in any desirable manner (e.g., locally, orally, systemically, intravenously, intraarterially, subcutaneously, or intrathecally). The invention also provides pharmaceutical compositions comprising the TGF-β inhibitor in a pharmaceutically acceptable carrier.

Such methods are not limited to a specific meaning for a therapeutically effective amount of TGF-β inhibitor capable of treating, delaying progression of, or reducing the severity of spinal cord injuries. In some embodiments, a therapeutically effective amount is an amount capable of inhibiting, delaying progression of, or reducing the severity of fibrotic scar formation at and/or around a site of spinal cord injury. In some embodiments, a therapeutically effective amount is an amount capable of inhibiting, delaying progression of, or reducing the severity of fibroblast differentiation of mesenchymal stromal/stem cells (MSCs) at and/or around a site of spinal cord injury.

In some embodiments, the subject is a mammalian subject (e.g., mouse, horse, human, cat, dog, gorilla, chimpanzee, etc.). In some embodiments, the subject is a human patient suffering from a spinal cord injury.

The invention also provides kits comprising an agent capable of inhibiting TGF-β activity at and/or around a site of spinal cord injury and instructions for administering the agent to an animal. The kits may optionally contain one or more other therapeutic agents.

Any of the agents described herein, in addition to configured for administration to a subject as a raw chemical, may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The agents and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

The following examples are illustrative, but not limiting, of the present invention. Other suitable modifications and adaptations of the variety of conditions and parameters normally encountered in clinical therapy and which are obvious to those skilled in the art are within the spirit and scope of the invention. As used herein, terms such as “our”, “we”, “I”, and similar terms, refers to the inventive entity for this invention.

### Example I

This example characterizes progression of ossification of spinal ligaments and hip joint ligaments of AS patients.

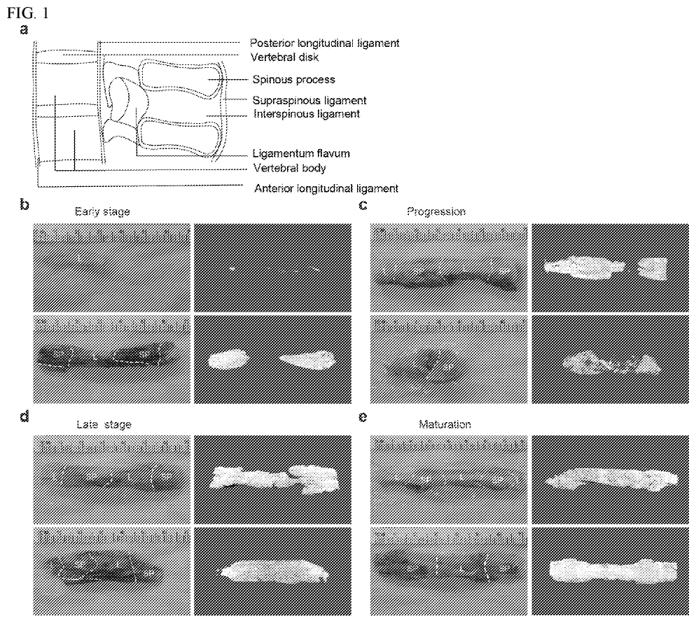

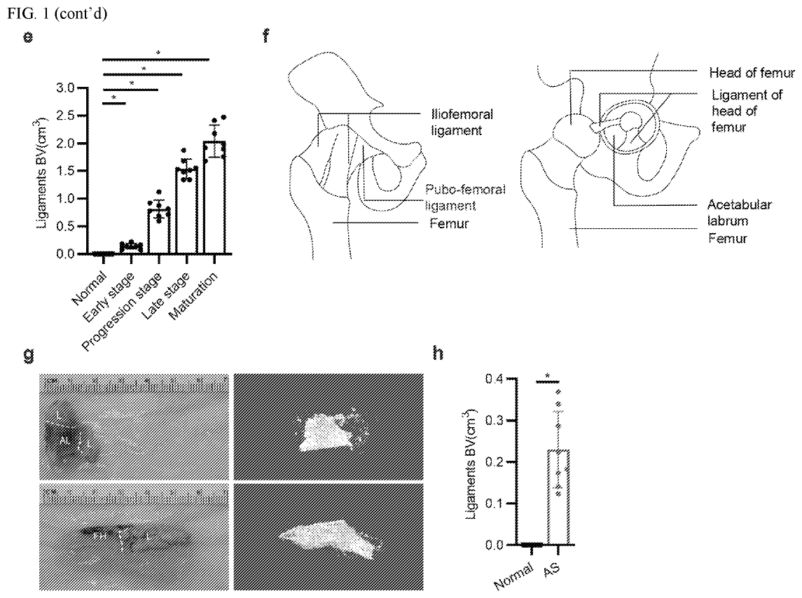

We have collected 37 surgery specimens of AS patients with fused spine or hip joints. These specimens were systemically scanned and analyzed by micro-computerized tomography (μCT), the illustration indicated the specimens we obtained from patients which included Interspinous ligament with Supraspinous ligament, with/without Ligamentum flavum, with/without Posterior longitudinal ligament (FIG. 1a). We observed AS progression of ossification in spinal ligaments. Particularly in interspinous ligaments since interspinous ligaments were kept in all the specimens we collected, the ossification could be seen with naked eyes at different stages: early stage starting ossification (FIG. 1b), progression stage with partial ossification (FIG. 1c), middle-late stage with incomplete ossification (FIG. 1d) and mature stage with complete ossification (FIG. 1e). μET analysis of these 4 different stages of the interspinous ligaments also revealed that the bone volumes were increased in progression from early to mature stage (FIG. 1e) while there were no ossification within normal ligaments. We also analyzed specimens of AS patients with fused hip joints, the illustration indicated the specimens we obtained from hip joints of patients included ligaments of head of femur which attached with either Head of femur or Acetabular labrum (FIG. 1G).We observed ossification in ligaments of hip joints and mCT analysis of these ligaments also revealed that there were ossification within these ligaments while there were no ossification within normal ligaments. As 3D reconstruction of μCT scan (FIG. 1b-e and h) showed, before ligaments became as completely bony structure, most of the interspinous ligaments in specimen were in the process of ossification. We therefore systematically analyzed whether spine fusion in the soft tissue between vertebral bodies such as interspinous ligaments, supraspinous ligaments, and the space of sacroiliac joints is a process of heterotopic ossification.

### Example II

This example demonstrates increased CD68⁺ macrophages and TGF-β activity at early inflammatory stage of AS spine.

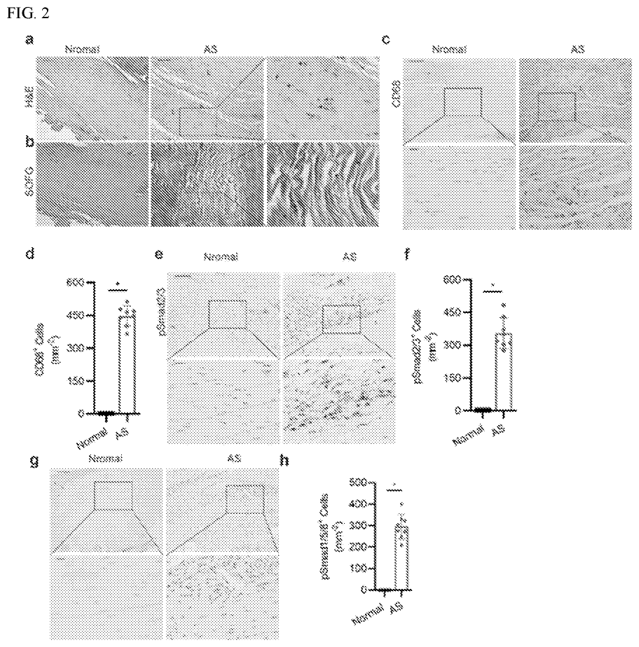

We first performed immunohistology analysis of the ligament specimen at early inflammatory stage of AS spine. After decalcification, the sagittal sections were prepared from the embedded interspinous ligament specimens. H&E staining of non-calcified area showed that the fibers were disorganized and infiltrated with more cells relative to normal control, shape of ligament cells turned to circle from fusiform (as indicated by black arrow) (FIG. 2a). Safranin O and fast green staining of sagittal sections of non-calcified area of AS interspinous ligaments showed that there were no calcification and cartilage formation relative to normal control (FIG. 2b). However, immunostaining demonstrate that significant increase of CD68⁺ macrophages in the AS interspinous ligament (FIG. 2c), indicating that inflammation when there was no calcification. Importantly, immunostaining of phosphorylated Smad2/3 (pSmad2/3), a downstream signaling transducer of TGF-β, revealed that the level of pSmad2/3⁺ cells, significantly increased in the inflammation area of AS ligaments (FIG. 2e). These results demonstrated that TGF-β activity was significantly increased at early inflammatory stage of AS. We also found higher levels of pSmad1/5/8 signaling in ligaments of AS patients when compared with normal control, suggesting active BMP signaling.

### Example III

This example demonstrates chondrogenesis in the interspinous ligaments of AS patients.

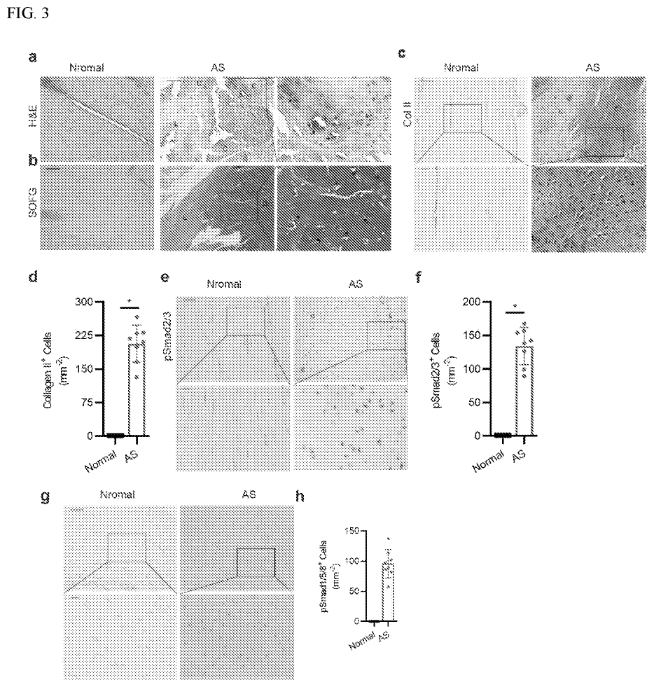

We have shown that increased TGF-β activity promotes chondrogenesis as an essential stage of HO. To examine whether spine fusion undergo a cartilage formation, we performed histological analysis of AS interspinous ligaments with limited calcification areas at progression stage as determined by μCT. H&E staining of sagittal sections of AS interspinous ligaments showed morphology of cartilage formation in the ligaments (FIG. 3a, black arrow). Safranin O and fast green staining of sagittal sections of AS interspinous ligaments confirmed cartilage formation with chondrocytes in the cartilage lacuna (FIG. 3b). Moreover, immunostaining of collagen II (Col II) for chondrocytes demonstrates that Col II⁺ cells were significantly increased in the AS interspinous ligaments relative to normal control, indicating chondrocyte differentiation and cartilage formation (FIG. 3c). Again, immunostaining of pSmad2/3 showed that level of pSmad2/3⁺ cells significantly increased in the AS interspinous ligaments, especially at chondrogenesis sites (FIG. 3e). These results revealed that AS patient spinal soft tissues undergo chondrogenesis in the AS interspinous ligaments prior to calcification. At this stage, the levels of pSmad1/5/8 signaling were also higher in ligaments of AS patients relative to normal control, but not as active as pSmad2/3 signaling.

### Example IV

This example demonstrates endochondral ossification in the bony interspinous ligaments of AS fused spine.

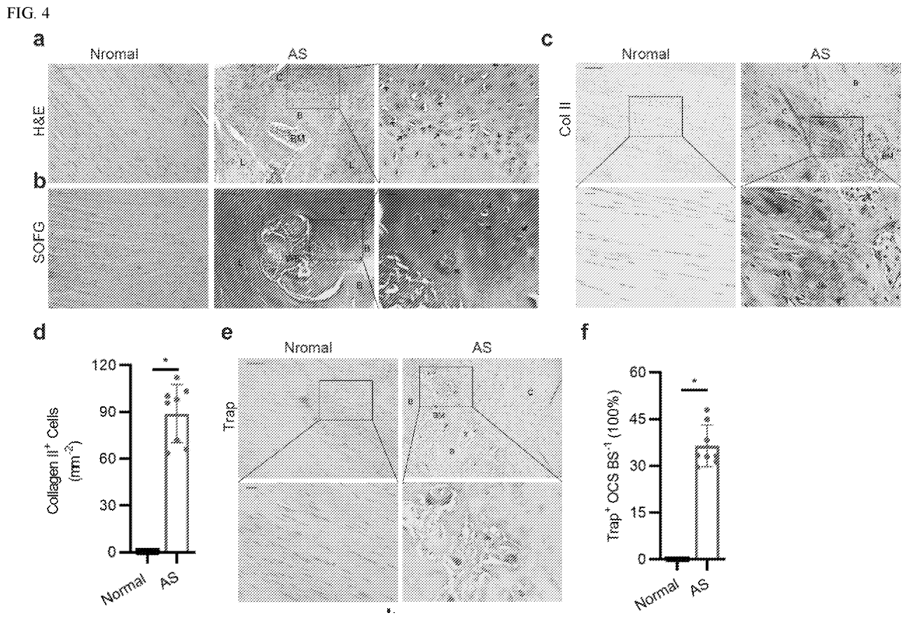

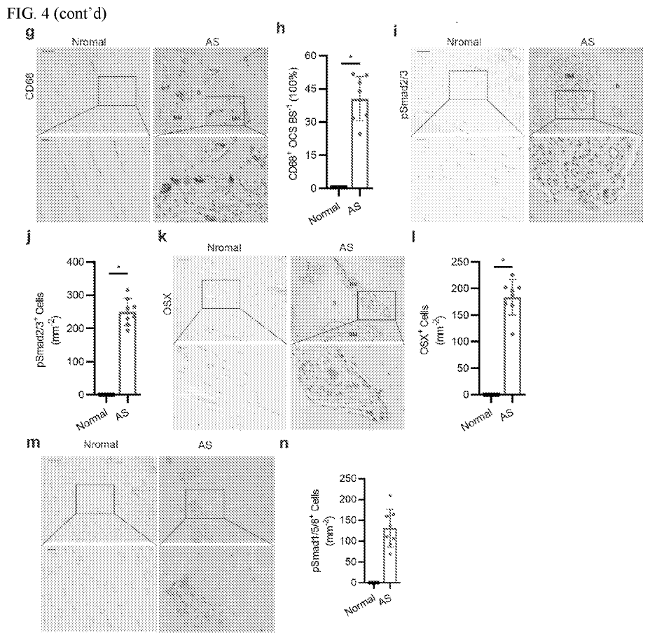

Chondrogenesis in the AS interspinous ligaments suggests that AS spine fusion is a process of HO. We then examined whether chondrogenesis at progression stage lead to endochondral ossification stage of HO. Interspinous ligaments at middle-late stage of AS were embedded and sections were prepared. H&E staining of sagittal sections of calcified area of AS interspinous ligaments revealed that new bone was formed with marrow surrounded by thick layers of cartilage (FIG. 4a), indicating endochondral ossification. Safranin O and fast green staining confirmed that calcified green stained area was surrounded by Safranin O red cartilage with chondrocytes and cartilage lacuna and that the boundary between cartilage and bone was clearly seen (FIG. 4b, indicated by black hollow arrow). Furthermore, immunostaining of collagen II demonstrated that large number of Col II⁺ cells were at cartilage sites adjacent to bone and marrow (FIG. 4c). Resorption of calcified cartilage by osteoclasts activates TGF-β to recruit osteoprogenitors as a process of endochondral bone formation in HO. Tartrate-resistant acid phosphatase (TRAP) staining for osteoclasts showed that there were large numbers of TRAP⁺ osteoclasts on the bone surface in the marrow resorption cavity (FIG. 4e). Like TRAP staining results, immunostaining of CD68 also showed large number of CD68⁺ osteoclasts on bone surface (FIG. 4g). Immunostaining of pSmad2/3 demonstrated that level of pSmad2/3 in the bone marrow significantly increased relative to normal control (FIG. 4i), suggesting osteoclast resorption in activation of TGF-β. Release of active TGF-β from osteoclast resorption recruits osteoprogenitors for new bone formation. Indeed, immunostaining of Osterix (OSX) showed that Osterix⁺ osteoprogenitors were lined on the bone surface (FIG. 4k). Noticeably, the Osterix⁺ osteoprogenitors and TRAP⁺ osteoclasts were not found in the same marrow areas, indicating uncoupled bone resorption and bone formation. Taken together, the results reveal that endochondral bone formation proceeded on calcified cartilage during progression of AS. pSmad1/5/8 signaling also activated in ligaments of AS patient at this stage, however, similar to chondrogenesis stage, its activity was not elevated significantly as pSmad2/3 signaling.

### Example V

This example demonstrates that uncoupled osteoclast resorption of fused bony ligaments drives progression of AS at mature stage.

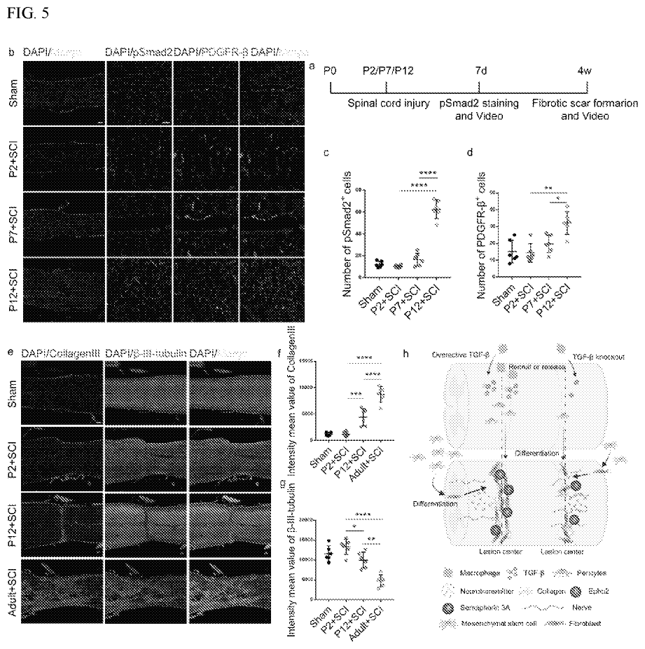

We have shown that osteoclast resorption activates TGF-β promotes HO progression even at middle-late and mature stage. Therefore, we examined whether fused bony spine at mature stage is still actively remodeled by osteoclasts for AS progression and pain sensory innervation. Bony interspinous ligaments at mature stage were confirmed by μCT and embedded for preparation of sections (FIG. 1e). Large number of giant osteoclasts on bone surface and Green stained bone formation were shown in H&E and Safranin O and fast green staining of mature AS ligaments (FIG. 5a, b). TRAP staining confirmed that large number of osteoclasts formed on the bone surface (FIG. 5c). Particularly, the size of these osteoclasts was much larger than those observed at progression stage. Immunostaining of CD68⁺ further showed that the osteoclasts were giant on the bone surface (FIG. 5e). Moreover, immunostaining demonstrated that high level of pSmad2/3 in the in the marrow cavity (FIG. 5g) and significant increase of Osterix+ osteoprogenitors at the bone surface (FIG. 5i). Osteoclast bone remodeling is often coupled with angiogenesis by secretion of PDGF-BB, specifically type H blood vessel formation in mice. Immunostaining showed that PDGF-BB level was significantly higher at the marrow cavity of mature AS ligament relative to control (FIG. 5k). Furthermore, immunostaining of CD31 and Emcn demonstrated significant increase of blood vessel formation (FIG. 5m,n). These results indicate that osteoclast remodeling of bony fused spine activates TGF-β in coupling new bone formation and angiogenesis to promote progression of AS. The results also suggest that formation of giant osteoclasts is still actively remodeling of bony fused spine to drive AS progression at mature stage. BMP signaling was also explored by testing pSmad1/5/8 signaling, the level of pSmad1/5/8 signaling was high in bone marrow of newly formed ectopic bone.

### Example VI

This example provides a discussion related to Examples I-V.

New bone formation along axial skeleton is the hallmark of AS post inflammation beginning in the sacroiliac joints. Anti-inflammation therapies such as anti-TNF-α antibody effectively reduce inflammation but are not able to slow stiffness and fusion in the spine of AS patients. In the experiments described during the course of developing embodiments for the present invention, we found that cartilage formation in the ligaments in the AS patients and chondrocytes were clearly detected in the cartilage lacuna as shown in our immunohistostaining. Moreover, collagen II (Col II) staining validated abundant Col II⁺ chondrocytes in the process of chondrogenesis within the ligaments. Importantly, Safranin O and fast green staining demonstrate that calcified tissue were observed adjacent to cartilage, indicating a process of endochondral ossification in the spine of AS patients. The finding of chondrogenesis in AS patients uncovers that cartilage formation acts as an intermediate stage to mediate ossification progression from inflammation microenvironment. Indeed, the level of active TGF-β significantly increased at inflammation and progression stages in the spine of AS patents. TGF-β has broad spectrum of functions including inflammation, cell migration, chondrogenesis, angiogenesis, epithelial mesenchymal transition, and remodeling of the new ECM (see, Wang X, et al., Nat Commun 9, 551 (2018); Zhen G, et al. Nat Med 19, 704-712 (2013); Medici D, Olsen B R. The role of endothelial-mesenchymal transition in heterotopic ossification. J Bone Miner Res 27, 1619-1622 (2012)). TGF-β plays an essential role in chondrocyte differentiation, cartilage formation and bone remodeling (see, Tang Y, et al. Nat Med 15, 757-765 (2009); Blaney Davidson E N, et al., Osteoarthr Cartil 15, 597-604 (2007)). We have shown that inflammatory environment with high levels of active TGF-β provides the conditions for cartilage formation as an intermediate phase leading to endochondral ossification in acquired HO. Thus, cartilage formation in the AS patients reveals that spine fusion experiences a similar process of acquired HO.

There are four stages of acquired HO as shown in our previous study: inflammation, chondrogenesis, osteogenesis, and maturation (see, Wang X, et al. Nat Commun 9, 551 (2018)). Chondrogenesis leads to osteogenesis in HO progression. Analysis of incomplete ossification ligaments of AS patients revealed that endochondral ossification occurred within the cartilage, in which chondrocytes underwent hypertrophy then calcification. Importantly, TRAP staining showed that many TRAP osteoclasts were found at the surface of bone cavities. Invasion and aberrant resorption by these giant CD68⁺ osteoclasts generated bone cavities like marrow (see, O'Brien E J O, et al., Int J Exp Pathol 93, 319-331 (2012); Wozney J M, et al. Science 242, 1528-1534 (1988)). TGF-β activated by osteoclast bone resorption recruits MSCs in coupling with new bone formation (see, Zhen G, et al. Nat Med 19, 704-712 (2013)). High levels of active TGF-β were detected in marrow cavity evidenced by large number of pSmad2/3⁺ cells. Active TGF-β is released by osteoclast bone resorption to couple bone resorption with formation (see, Tang Y, et al. Nat Med 15, 757-765 (2009)). Indeed, abundant OSX⁺ progenitors were detected in the marrow cavity, but they were not located at osteoclast resorptive sites, rather dispersed at areas with active bone formation. This is a typical uncoupled bone resorption and formation as we have observed HO (see, Wang X, et al. Nat Commun 9, 551 (2018)). Bone formation is always associated with angiogenesis such as type H vessel formation in mice (see, Xie H, et al. Nat Med 20, 1270-1278 (2014); Kusumbe A P, et al., Nature 507, 323-328 (2014); Ramasamy S K, et al., Nature 507, 376-380 (2014)). During remodeling, TRAP⁺ lineage osteoclastic cells secrete PDGF-BB for angiogenesis and Netrin-1 for sensory innervation (see, Xie H, et al. Nat Med 20, 1270-1278 (2014); Zhu S, et al. J Clin Invest 129, 1076-1093 (2019); Ni S, et al. Nat Commun 10, 5643 (2019)). Very abundant blood vessels were observed during HO progression. In AS patients, highly elevated levels of PDGF-BB in marrow cavity as the most upstream regulator for blood vessel formation. Back pain is one of the main clinical manifestations in AS (see, Zhu W, et al. Bone Res 7, 22 (2019)) and CGRP⁺ nerve fibers were detected in the bony ligaments of AS patients. Giant osteoclasts are likely responsible for the PDGF-BB-induced blood vessels formation and CGRP⁺ nerve innervation.

There have been over 100 clinical trials for AS but there is still no effective treatment for reduction of both disease activity and structural progression. NSAIDs as the first-line treatments for active AS, they do relief the inflammatory back pain and improve the function activity in AS patients (see, Roux C. Osteoporos Int 22, 421-433 (2011)). However, continuous use of NSAIDs showed no benefits on slow of AS any clinical aspect (see, Campagna R, et al. A J R Am J Roentgenol 192, 987-995 (2009)). TNF-α antibody or inhibitors are effective on reduction of inflammation in AS patients, but no significant effect on halting of osteogenesis in progression of spine fusion. In addition, predictors for a good response to TNF-α inhibitors make their usage is limited including male sex, a relative short disease duration, HLA-B27 positive, patient age ≤40 years, the absence of enthesitis, a good functional status as well as a high CRP level. Therefore, neither NSAIDs treatment nor TNF-α inhibitors could halt the progression of structural changes in AS (see, Schett G, et al., Best Pract Res Clin Rheumatol 24, 363-371 (2010); Poddubnyy D, et al., Expert Opin Biol Ther 13, 1599-1611 (2013); Keifer J, et al. EMBO J 10, 4025-4031 (1991)). Interestingly, AS clinical trials with bisphosphonates showed better effect on improvement of symptoms and spine structure change in comparison with TNF-a antibody therapy.

The findings of AS in current study provides an explanation for the results of those clinical trials. Progression of AS is a process of acquired HO and autoimmune inflammation generates environment for cartilage formation as an initiation of ossification. Apparently, inhibition of inflammation does not effectively halt ossification. Particularly, once cartilage has been formed, ossification will be continuously remodeled by osteoclast bone resorption and inflammation condition will be no longer required. At mature stage, interspinous ligament become complete bone structure in μCT scan. However, histological staining demonstrates that there are still large number of giant osteoclasts with high level of active TGF-β in the remodeling marrow of bony interspinous ligament. The aberrant osteoclast resorption drives the progression ossification. This explains inhibition of osteoclast activity by bisphosphonates could slow new bone formation. Our findings also suggest that inhibition of HO such as cartilage formation, osteoclast resorption or TGF-β activity could be effective therapy for AS.

### Example VII

This examples provides the materials and methods for the experiments described in Examples I-VI.

### Human Subjects

The study was approved by Johns Hopkins University and Peking Union Medical College Hospital review board. All pathology tissues were obtained with written informed consent from individuals. Pathological specimens were collected from 37 Ankylosing spondylitis patients (34 male and 3 female, no Fibrous dysplasia of bone or other blastic bony metastases alone, nonsmoking individuals, age ranging from 26 to 64 years, disease duration ranging from 10 to 40 years) who met the 1984 modified New York criteria after spinal wedge osteotomy. Heterotopic ossification was firstly accessed by computed tomography scan (CT scan) of each patient before enrollment and identified by Micro-CT scan (μCT scan) after collection. Healthy specimens were collected form 13 patients (8 male and 3 female, no Fibrous dysplasia of bone or other blastic bony metastases alone, nonsmoking individuals, age ranging from 23 to 65 years) with total hip replacement or spinal fracture during surgery. The obtained specimens including Hip joint (femoral head with ligaments and acetabular labrum with ligaments; 5 from healthy patients and 5 from AS patients after total hip replacement), Spinous process with interspinous ligament, supraspinous ligament and/or ligamentum flavum (8 from healthy patients after traumatic surgery and 32 from AS patients after spinal wedge osteotomy) . . . Spinal ligaments at Early stage (Disease duration ranging from 10 to 18 years; 6 male and 2 female), progression stage (Disease duration ranging from 10 to 21 years; 7 male and 1 female), late stage (Disease duration ranging from 15 to 27 years; 8 male) or maturation stage(Disease duration ranging from 20 to 40 years; 8 male) were defined based on the μCT results.

### MicroCT Analysis

Human tissues including hip joint (femoral head with ligaments and acetabular labrum with ligaments), spinous process with interspinous ligament, supraspinous ligament and/or ligamentum flavum from both healthy patients and AS patients were fixed overnight in 10% formalin and analyzed by high-resolution μCT (Skyscan1172, Skyscan1272). The scanner was set at a voltage of 60 kV and a resolution of 9 μm per pixel. Images of PCT were used to perform 3D histomorphometric analyses. The region of interest was defined to cover the whole PCT compartment (Femoral head or acetabular labrum with ligaments, single spinous process or joint spinous process with interspinous ligament, supraspinous ligament and/or ligamentum flavum). The images were reconstructed by NRecon v1.6, analyzed by CTAn v1.9 and visualized using 3D model visualization software, CTVol v2.0.

### Histochemistry

The blocks were sectioned at 4 μm by using a Paraffin Microtome (for paraffin blocks). We processed 4-μm-thick sections of bone for H&E staining and safranin O (Sigma-Aldrich, S2255) and fast green (Sigma-Aldrich, F7252) staining. We performed H&E staining and safranin O and fast green staining used paraffin sections. The sections were de-waxed at first and washed 3 times with PBS then. For H&E staining, sections were Stained nuclei with hematoxylin (Sigma-Aldrich, H9627) for 5 minutes and following rinsing in running tap water for 10 seconds, then differentiated with 0.3% acid alcohol for 10 seconds, rinsing in running tap water for 1 minutes again, followed by counterstaining with Eosin Y (Thermo-fisher, 7111) for 1 minutes. For safranin 0 and fast green staining, sections were counterstaining with fast green for 5 minutes and differentiated with 1% acetic acid for 10 seconds after that. Then counterstaining with safranin 0 for 5 minutes. TRAP staining was processed following the manufacturer's protocol (Sigma-Aldrich, 387A-1KT), followed by counterstaining with Methyl Green (Sigma-Aldrich, M884).

### Immunohistochemistry Histomorphometry

The blocks were sectioned at 4 μm or 60 μm (for CD31 and Emcn immunofluorescent staining) intervals using a Microm cryostat (for frozen blocks) or a Paraffin Microtome (for paraffin blocks). For immunohistochemistry staining, after de-waxed, paraffin sections were heated to 99° C. for 15 minutes in Target Retrieval Solution (Dako, S1699) for antigen retrieval, followed by 30 minutes at room temperature, then sections were rehydrated. After washing three times with PBS, the tissue sections were incubated with blocking solution for 1 hour, then we incubated sections with primary antibodies to human pSmad2/3 (Santa Cruz Biotechnology Inc., sc-11769, 1:50), human CD68 (Invitrogen, MA5-13324,1:100), human Collagen II (Invitrogen, MA5-12789,1:100), human Osterix (Abcam, ab22552, 1:100), human PDGF-BB (Abcam, ab21234, 1:50), human Endomucin (Santa Cruz, V.7C7, sc-65495,1:50) at overnight at 4° C. in a humidified chamber followed by 1 hour at room temperature. The sections were washed three times with Tris-buffered saline, then we incubated slides with secondary antibodies in blocking solution for 1 h at room temperature and used Chromogenic Substrates (Dako, K3468) to detect the immunoactivity subsequently, at last we counterstained with hematoxylin (Sigma-Aldrich, H9627). For immunofluorescence staining, after incubated at 37° C. for 30 minutes, we washed sections three times with PBS, then sections were incubated with blocking solution for 1 hour, after that sections were incubated with primary antibodies to human CD31 (Abcam, ab28364, 1:100), human Endomucin (Santa Cruz, V.7C7, sc-65495,1:50) at overnight at 4° C. in a humidified chamber followed by 1 hour at room temperature. The sections were washed three times with Tris-buffered saline, then we use secondary antibodies conjugated with fluorescence at room temperature for 1 hour, while avoiding light and mounted on slides with ProLong Gold Mounting Reagent with DAPI (Life Technologies, P36935). We used isotype-matched controls, such as polyclonal rabbit IgG (R&D Systems, AB-105-C), polyclonal goat IgG (R&D Systems, AB-108-C), and monoclonal rat IgG2A (R&D Systems, 54447) under the same concentrations and conditions as negative controls.

### Histomorphometry

We process imaging samples by using an Olympus DP71 microscope. We analyzed human specimens according to five sequential sections per stain and used anatomic landmarks to ensure comparability included the presence of bone marrow and bone matrix. Also, serial sagittal sections of the HO lesions were obtained. The numbers of positively stained cells in five random visual fields in five sequential sections per specimen in each group were counted and they were normalized to the number per millimeter of adjacent bone surface (for TRAP staining quantification) or per square millimeter in HO area. We conducted the Quantitative analysis with OsteoMeasureXP Software (OsteoMetrics, Inc.). For CD30⁺ vessels and Emcn⁺ vessels quantification, we calculated the area of red color (CD31⁺) and green color (Emcn⁺) in the whole HO site of each slide in three sequential sections per specimen in each group and normalized to that of control specimen (set to 1).For quantification of chondrocytes, we calculated all the cells in brown (COL II+) area and considered them as COLII+ chondrocytes. Quantifications were performed using ImageJ 1.53c software.

### Statistics

All statistical analyses were performed by SPSS 15 software. The data are presented as the mean±S.D. We performed comparisons using unpaired, two-tailed Student's t-test for separately comparison of morphometric analysis of inflamed stage, chondrogenesis stage, osteogenesis stage or maturation stage of healthy human patients and AS human patients. The level of significance was set at p<0.05. All inclusion/exclusion criteria were pre-established, and no samples were excluded from the analysis. No statistical method was used to predetermine the sample size. The experiments were randomized. The investigators were not blinded to allocation during experiments and outcome assessment.

### Example VIII

This example describes a complete recovery from SCI in neonatal mice with no detectable active TGF-β and fibrotic scar formation.

Neonatal mice do not exhibit fibrotic scar formation within 7 days after SCI. We examined if active TGF-β is involved in fibrotic scar formation in neonatal mice after SCI. In brief, the spinal cord was crushed with forceps to generate SCI in neonatal mice on day 2, 7 and 12 after birth. SCI tissues were harvested, and sections were prepared (FIG. 5A). Immunostaining of pSmad2 showed that pSmad2⁺ cells were not detectable in the SCI site of postnatal day 2 and 7 (P2 and P7) mice as they were in sham-operated neonatal mice on day 7 (FIG. 5B, C), whereas the number of pSmad2⁺ cells was significantly increased in postnatal day 12 (P12) mice. All 3 members of the TGF-β family, comprising TGF-β1, 2, and 3, are expressed in mammals but not expressed in aquatic vertebrates and amphibians in the process of evolution. Importantly, non-mammal vertebrates do not face the same obstacles as mammals when it comes to recovery from SCI⁴⁶⁴⁸. We posited that if no fibrotic scar formation occurred in neonatal mice, recovery from SCI could be expected to occur as it does in amphibians.

We examined the recovery of P2 and P12 mice at 7 days and 1 month after SCI by video recording as the behavior tests can not be conducted in neonatal mice. Interestingly, the P2 mice were almost completely recovered at 7 days and 1 month after SCI, similar to the sham littermates. However, the hindlimbs of P12 mice were still paralyzed at 1 month after SCI. Moreover, co-immunostaining of PDGFR-β, a marker for MSCs, showed that PDGFR-β was expressed similarly in SCI P2 and P7 mice, and sham P7 mice, but significantly increased in P12 mice after SCI (FIG. 5B, D), suggesting fibrotic scar formation on day 12.

Interestingly, immunostaining of collagen III demonstrated no fibrotic scar formation in SCI mice, similar to sham mice, and clear fibrotic scar formation on day 12 with a smaller size relative to adult SCI fibrotic scar (FIG. 5E, F). Consistently, immunostaining of β-III-tubulin showed nerve growth through the SCI area with no scar in P2 mice, similar to sham mice (FIG. 5E, G), whereas the fibrotic scar blocked nerve growth through the SCI area in P12 mice. These results suggest that elevated active TGF-β-induced fibrotic scar inhibits recovery of SCI.

### Example IX

This example demonstrates that systemic injection of TGF-β-neutralizing antibody attenuated fibrotic scar formation and improved functional recovery in SCI mice.

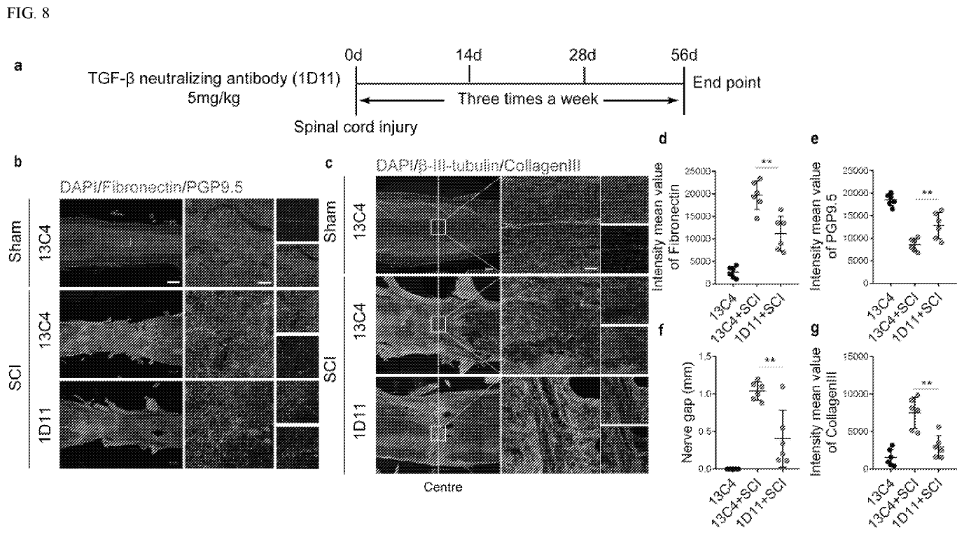

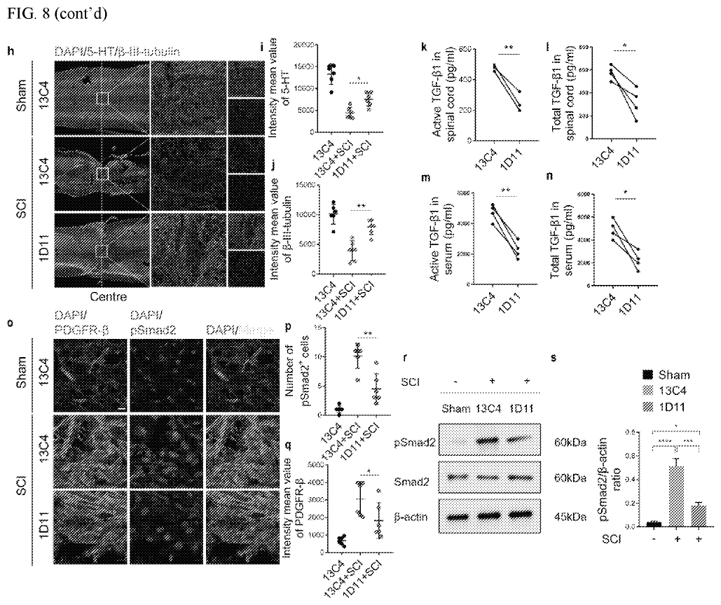

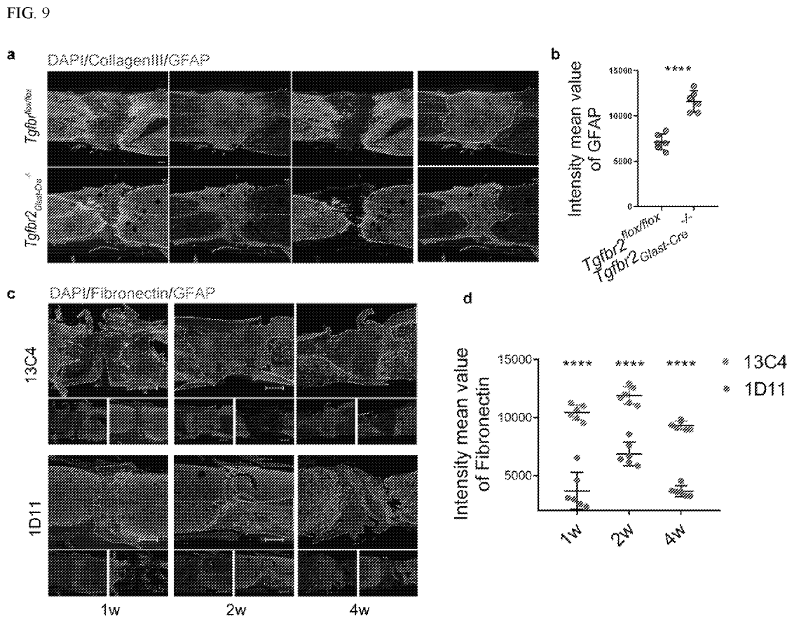

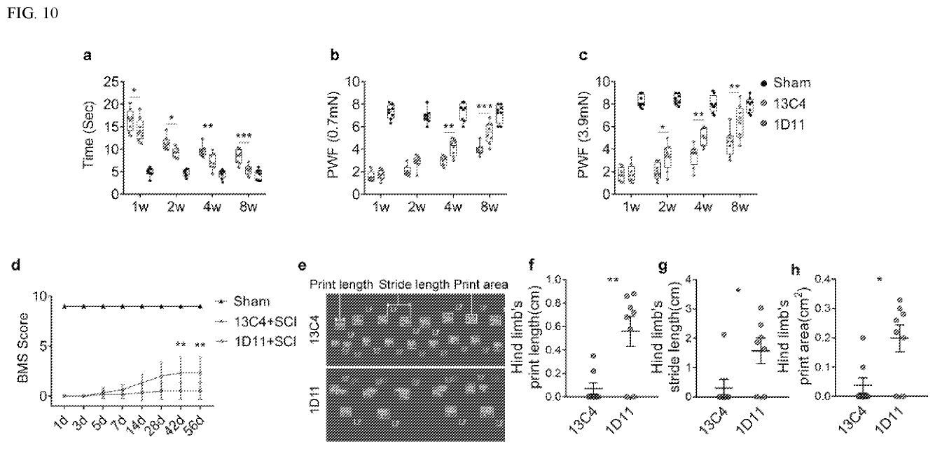

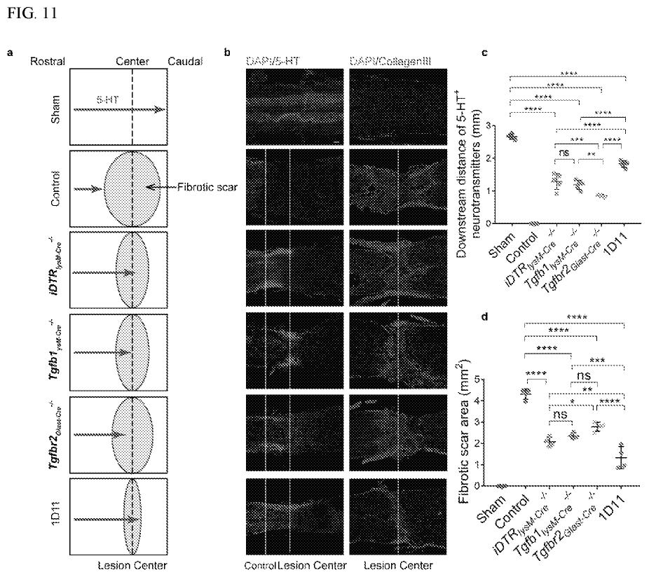

We next examined whether inhibition of TGF-β activity attenuates fibrotic scar formation and improves neurological functional recovery in adult mice. A TGF-β-neutralizing antibody (1D11) or a control antibody of an identical immunoglobulin G (IgG) complex lacking any TGF-β-binding capabilities (13C4) was injected intravenously 3 times a week as indicated in FIG. 8A. Co-immunostaining of PGP9.5 with fibronectin and collagen III with β-III-tubulin further confirmed that decreased fibrotic scar formation and a narrowed nerve gap with injection of 1D11 relative to control antibody 13C4 (FIG. 8B-G), and glial scar with abundant GFAP⁺ astrocytes was significantly increased (FIG. 9C, D). Moreover, injection of TGF-β-neutralizing antibody 1D11 significantly increased levels of serotonin neurotransmitter across injury site and nerve fibers at the injury area by co-staining 5-HT with β-III-tubulin (FIG. 8H-J). As expected, the concentrations of active and total TGF-β1 in serum and spinal cord decreased significantly with injection of 1D11 (FIG. 8K-N). Importantly, the number of pSmad2⁺ PDGFR-β⁺ pericytes almost diminished in 1D11 injection mice (FIG. 8O-Q), which was confirmed in Western blot analysis (FIG. 8R, S). Again, functional recovery measured using different behavior tests, including manual von Frey tests, hot plate test, BMS score, and gait analysis of the hindlimb, improved significantly in mice injected with 1D11 relative 13C4 control mice (FIG. 10). Most of the parameters related to fibrotic scar and functional recovery improved more in 1D11 injection mice relative to the other knockout mice (FIG. 11). Macrophages are likely not the sole source of active TGF-β. Collectively, systemic injection of 1D11 validates that excessive activation of TGF-β induces fibrotic scar formation and prohibits functional recovery in SCI mice.

### Example X

This example demonstrates that single-cell RNA sequencing showed elevated active TGF-β-induced fibroblast differentiation of MSCs in SCI mice.

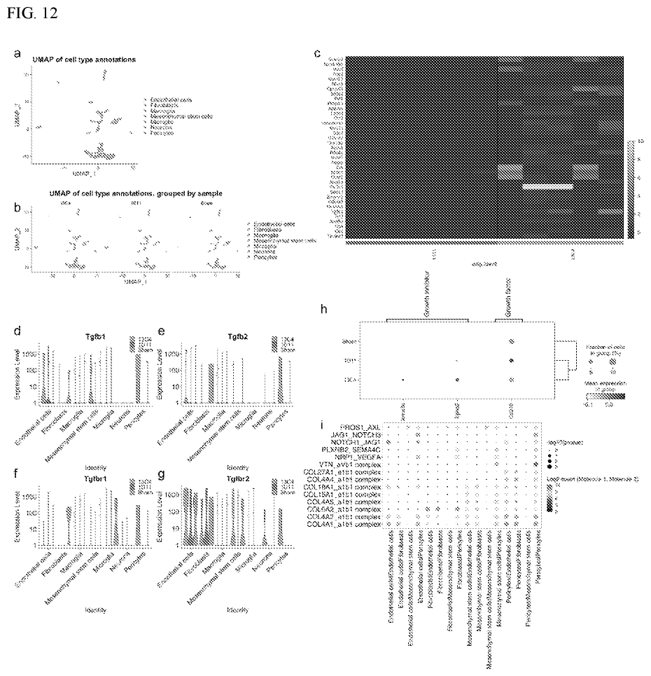

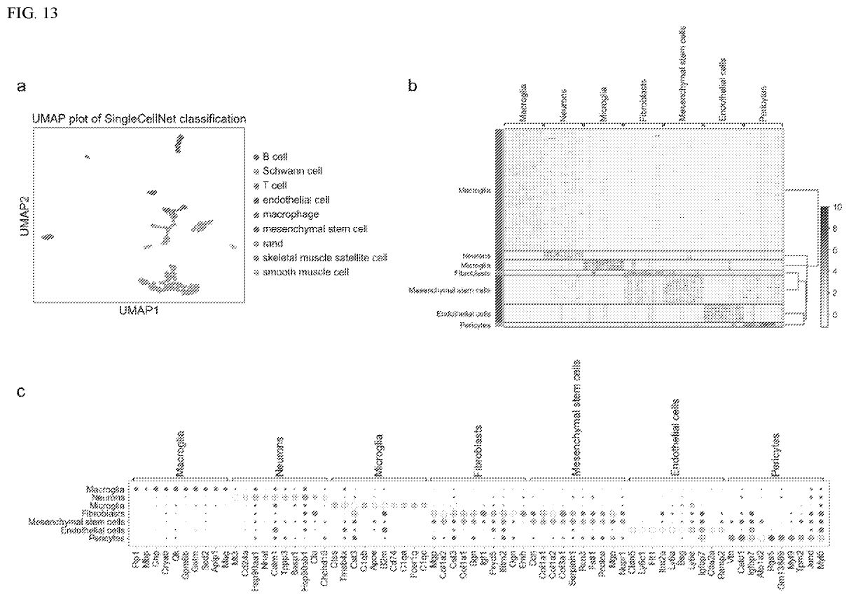

We then used single-cell RNA-sequencing technology to investigate the type of cells present at the fibrotic scar in SCI. TGF-β neutralizing antibody 1D11 or identical IgG complex lacking any TGF-β-binding capabilities 13C4 was injected intravenously in SCI mice with sham-operated mice as control. The SCI tissues were harvested at day 3 post injection and single cells were prepared for sequencing. After quality control, we obtained high-quality RNA-sequencing results from these three SCI single cell samples. Using a combination of automated cell typing and differential expression, we identified endothelial cells, fibroblasts, mesenchymal stem cells (MSCs), pericytes, microglia and neurons in all samples (FIG. 12A; FIG. 13A-C).

Enrichment analysis showed that the expression of downstream signaling genes was significantly increased in SCI treated with control 13C4 antibody relative to sham group, and almost all elevated expression of TGF-β downstream genes in the 13C4 group was significantly inhibited with injection of 1D11 (FIG. 12C). Moreover, expression of TGF-β ligands and receptors Tgfb1, Tgfb2, Tgfbr1 and Tgfbr2 was elevated in pericytes. MSCs, endothelial cells, fibroblasts, and microglia of 13C4 group relative to sham group, and significantly inhibited with injection of 1D11 (FIG. 12D), indicating that these cell types are specifically responsive to inhibition of TGF-β signaling as potential target for fibrosis. These results collectively support the conclusion that TGF-β pathway activity is significantly enhanced in the 13C4 group and identify putative cell types with key roles in TGF-β-induced fibrotic scar formation following SCI.

Next, we interrogated the expression of known axon growth inhibitors and growth factors to test the hypothesis that inhibition of TGF-β signaling could encourage axon growth by modulating the secretion of these mediators. The mean expression of several known axon growth inhibitors such as Sema3a and Ephb2 was found to be lower in the 1D11 group as compared to the 13C4 group while mean expression of axon growth factor CD248 was found to be higher in the 1D11 group as compared to the 13C4 and Sham groups, indicating that suppressing TGF-β signaling may contribute to axon regrowth (FIG. 12I).

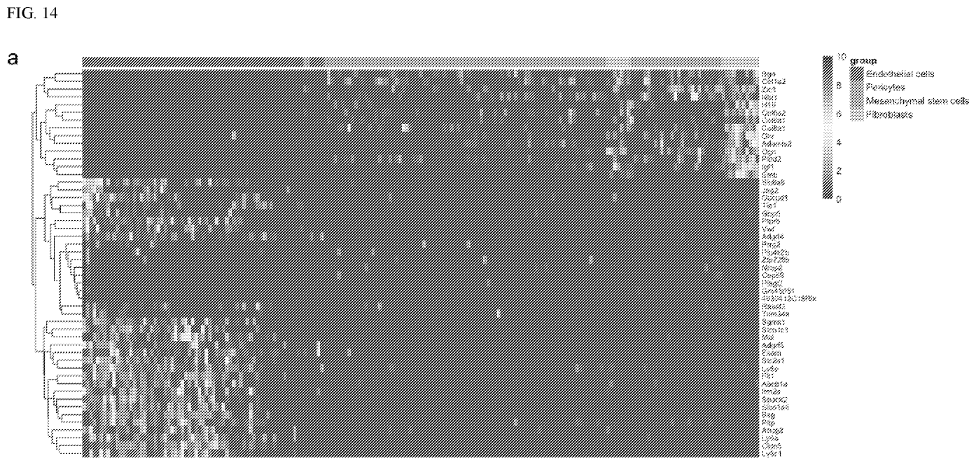

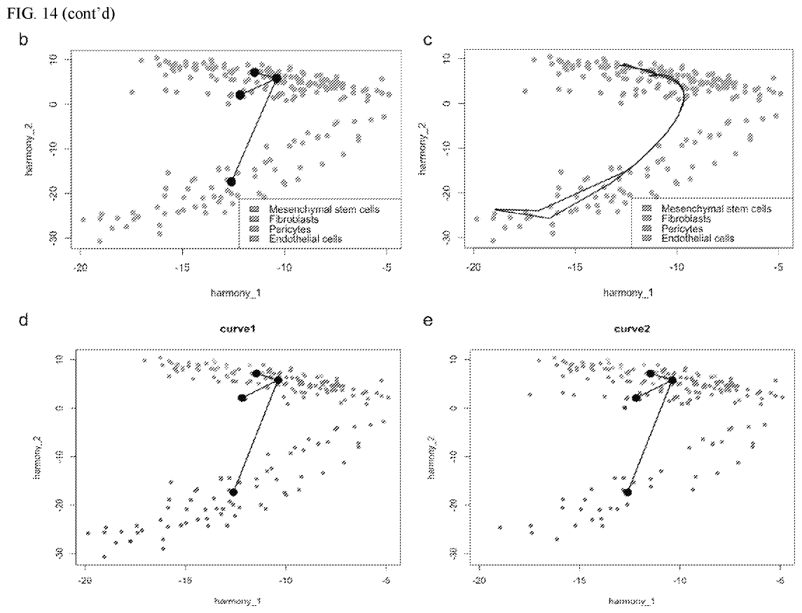

To determine the relationship between TGF-β-responsive cell types of interest, we performed trajectory analysis to reconstruct putative lineages connecting developmentally related cell types. We also performed a ligand-receptor analysis using CellPhoneDB to identify significant ligand-receptor interactions between cell types of interest. Because fibrotic scar was formed in 7 days post SCI as shown above and single cell preparation was impossible once scar is formed, samples for scRNAseq analyses were harvested 3 days post SCI when fibroblast differentiation is still in the middle stage. Thus, we performed lineage trajectory analysis on fibroblasts as well as MSCs, pericytes, and endothelial cells by computing pseudotime and revealed that MSCs give rise to fibroblasts at day 3 post SCI (FIG. 14A-E). Trajectory analysis returned two putative lineages: one representing a possible endothelial-to-mesenchymal transition of endothelial cells to fibroblasts through an MSC intermediate, and the other representing the differentiation of pericytes to fibroblasts through an MSC intermediate (FIG. 14B-E). Analysis of dynamically expressed genes along pseudotime of the endothelial cell lineage revealed gene expression changes indicative of endothelial-to-mesenchymal transition, such as loss of tyrosine kinase with immunoglobulin like and EGF like domains 1 (Tiel)⁴³ and von willebrand factor (vWF)⁴⁴ expression in endothelial cells and expression of collagen synthesis genes in fibroblasts (FIG. 14A). CellPhoneDB analysis further identified significant interactions between relevant ligand-receptor pairs with known roles in fibrosis, such as collagen-integrin binding and NOTCH-JAG^{∵} interactions amongst fibroblasts, MSCs, pericytes and endothelial cells (FIG. 12J). Taken together, our single-cell RNA-sequencing confirms enriched TGF-β downstream signaling in the control antibody treated sample, reveals enhanced cell-cell signaling in MSCs, fibroblasts, and pericytes, and may indicate a possible role for endothelial-to-mesenchymal transition in fibrotic scar formation.

### Example XI

This example demonstrates that a conditional knockout of TGF-β1 in the macrophage lineage cells reduced fibrotic scar formation in SCI mice.

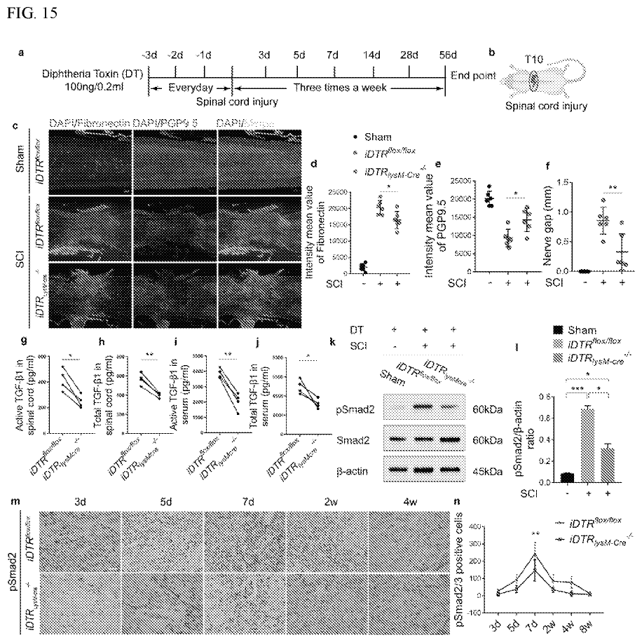

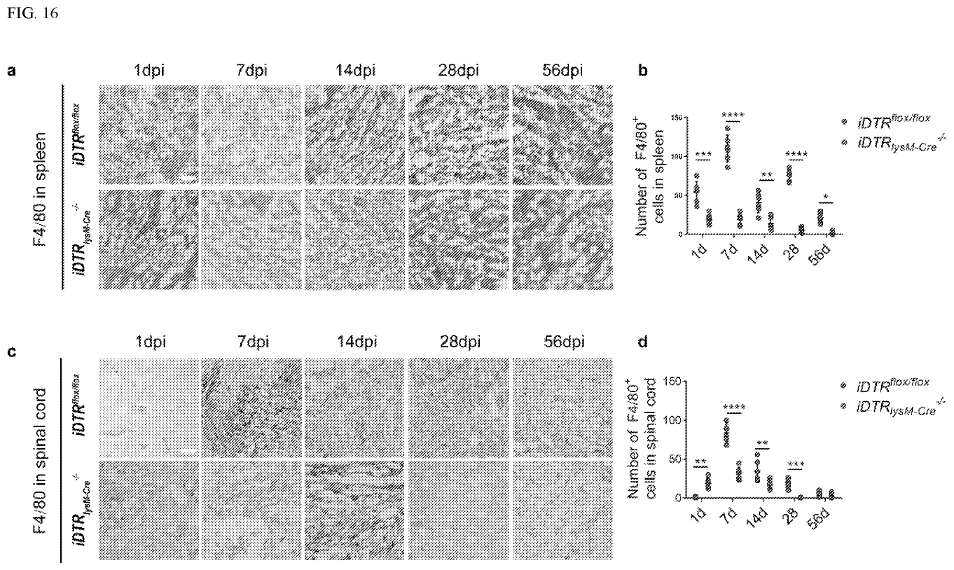

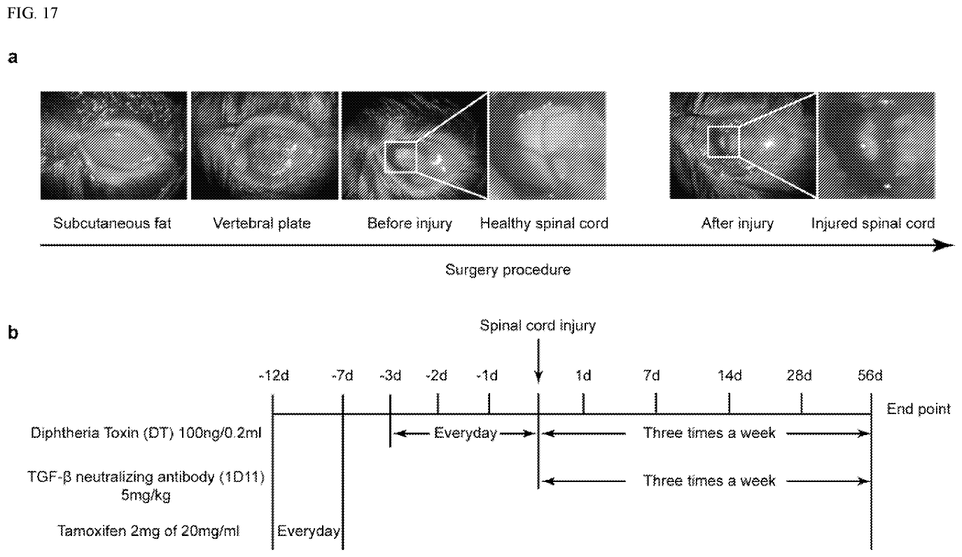

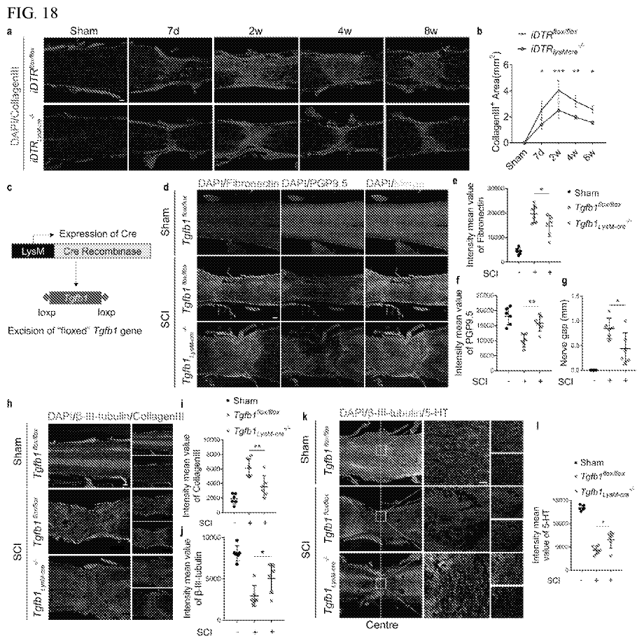

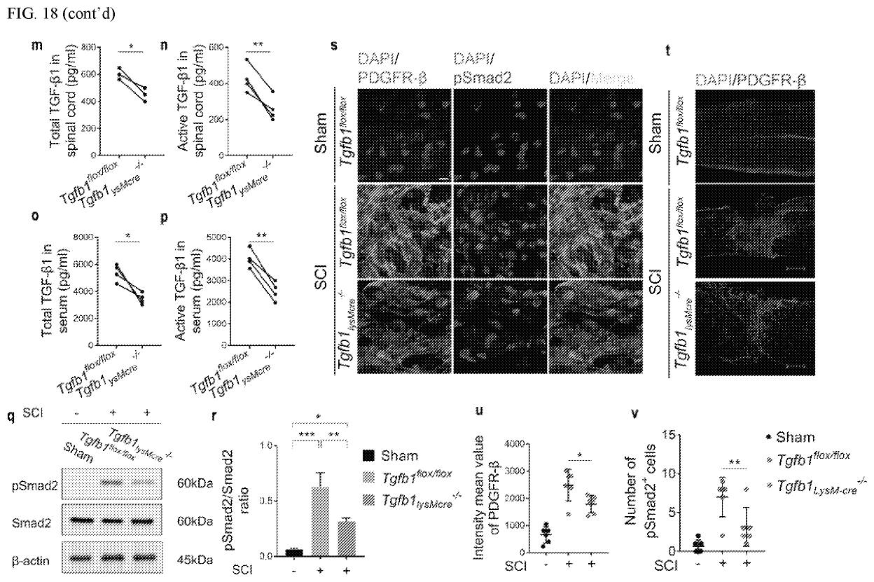

To investigate the source of the cells responsible for elevated active TGF-β, we crossed LysM-cre mice with iDTR^{flox/flox} mice to generate iDTR_{LysM-cre}^{-/-}, in which macrophage/monocyte lineage cells are selectively eliminated with injection of diphtheria toxin (DT) daily for 3 days (FIG. 15A). Efficient inhibition of TGF-β activity in iDTR_{LysM-cre}^{-/-} mice was evaluated by F4/80 staining of the spleen and spinal cord at different time points (FIG. 16). The spinal cord was then crushed with forceps to generate the mouse SCI model at day 0, and SCI tissues were harvested at day 3, 5, 7, 14, 28 and 56 (FIG. 15A, B; FIG. 17A)³⁴ . Sections of SCI tissues were prepared and immunostained for collagen III, a major component of fibrotic scar. The results showed that the collagen III-stained fibrotic scar size is increased significantly, peaking at 2 weeks after SCI, and continued to decrease in SCI iDTR^{flox/flox} mice, whereas fibrotic scar formation decreased significantly in SCI iDTR_{LysM-cre}^{-/-} mice relative to SCI iDTR^{flox/flox} mice (FIG. 18A, B). Moreover, co-immunostaining of protein gene product 9.5 (PGP9.5) and fibronectin, a fibrotic scar marker (FIG. 18B), further demonstrated that fibrotic scar decreased significantly in SCI iDTR_{LysM-cre}^{-/-} mice (FIG. 15C, D). The density of PGP9.5⁺ nerve fibers in the injured area increased significantly, and the nerve gap between the two sides of the injury site decreased markedly in SCI iDTR_{LysM-cre}^{-/-} mice relative to their SCI iDTR^{flox/flox} littermates (FIG. 15C, E, F). Importantly, the concentrations of active and total TGF-β1 in serum and spinal cord were significantly decreased in SCI iDTR_{LysM-cre}^{-/-} mice relative to SCI iDTR^{flox/flox} mice (FIG. 15G-J). Western blot confirmed that the phosphorylation of Smad2 were downregulated in SCI iDTR_{LysM-cre}^{-/-} mice (FIG. 15K, L). Indeed, the number of phosphorylated Smad2⁺ cells (pSmad2⁺), a TGF-β downstream signaling transducer, was significantly lower in iDTR_{LysM-cre}^{-/-} mice relative to iDTR^{flox/flox} mice. The number of pSmad²⁺ cells peaked 7 days after SCI and gradually returned to baseline by 8 weeks (FIG. 15M, N). Taken together, these results suggest that macrophages promote fibrotic scar formation through activation of TGF-β signaling.

To determine whether activation of TGF-β by macrophages induces fibrotic scar formation, we crossed LysM-cre mice with Tgfb1^{flox/flox} mice to generate Tgfb1_{LysM-cre}^{-/-} mice, in which macrophage/monocyte lineage cells no longer express TGF-β1 (FIG. 18C). Co-immunostaining of PGP9.5 with fibronectin showed significantly decreased fibrotic scar formation and increased density of nerve fibers in the lesion area of SCI Tgfb1_{LysM-cre}^{-/-} mice relative to Tgfb1^{flox/flox} mice (FIG. 18D-F). Specifically, the fibrotic scar area decreased significantly, and the gap between nerve fibers at the lesion center narrowed significantly (FIG. 18D, G). The results were confirmed with co-immunostaining of collagen III and 5-HT with β-III-tubulin, respectively (FIG. 18H-L). Importantly, the concentrations of active and total TGF-β1 in serum and spinal cord were significantly decreased in Tgfb1_{LysM-cre}^{-/-} mice (FIG. 18M-P). Western blot analysis confirmed that the number of pSmad2⁺ cells was significantly decreased in Tgfb1^{-/-} mice (FIG. 18Q, R). Moreover, co-immunostaining of pSmad2 with platelet-derived growth factor receptor-β (PDGFR-β), a marker of pericytes, showed that pSmad2⁺ cells were primarily PDGFR-β⁺ pericytes in SCI Tgfb1^{flox/flox} mice, and that the number of pSmad2⁺ PDGFR-β⁺ cells was significantly decreased in Tgfb1_{LysM-cre}^{-/-} mice, given that TGF-β promotes differentiation of PDGFR-β⁺ pericytes to fibroblasts (FIG. 18S-V).

### Example XII

This example demonstrates that a conditional knockout of Tgfb1 in macrophage lineage cells promoted functional recovery in SCI mice.

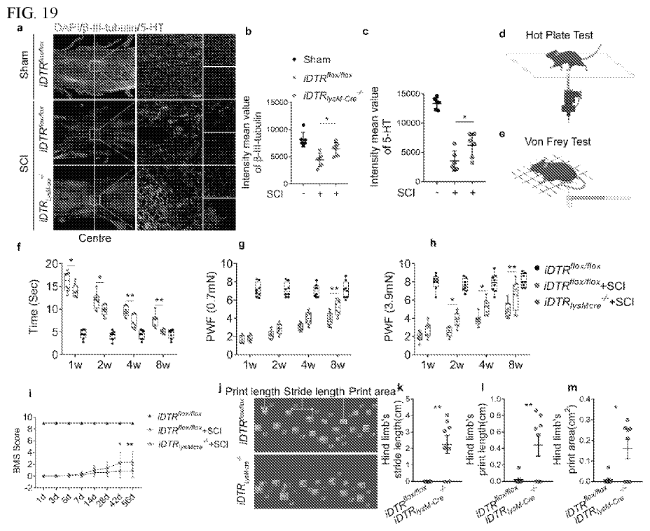

We then investigated whether the decreased fibrotic scar formation improved functional recovery after SCI. Given that serotonin-mediated innervation and the availability of 5-HT in the spinal cord are critical for the restoration of hindlimb motor function after SCI³⁵⁻³⁷, we performed co-immunostaining of 5-HT with β-III-tubulin, a neuron-specific cytoskeleton protein^{38,39}. The results showed β-III-tubulin-stained neuron fibers were significantly increased in the lesion area of iDTR_{LysM-cre}^{-/-} mice relative to iDTR^{flox/flox} mice (FIG. 19A, B). The concentrations of serotonin were higher at the upper site of the crush lesion and more neurotransmitters passed the lesion center in iDTR_{LysM-cre}^{-/-} mice (FIG. 19A, C). This observation suggests that decreased scar formation permitted neurites and neurotransmitters to cross the site of injury. The functional recovery in SCI iDTR_{LysiM-cre}^{-/-} mice compared with SCI iDTR^{flox/flox} mice was measured using manual hot plate test (FIG. 19D, F), von Frey test (FIG. 19E, G, H), Basso Mouse Scale (BMS) score (FIG. 19I), and gait analysis (FIG. 19J-M) on hindlimb function, including longer stride length, longer print length, and larger print area (FIG. 19K-M). Collectively, these results indicate that elevated TGF-β0 signaling in pericytes induces fibrotic scar formation to impede sensorimotor recovery after SCI.

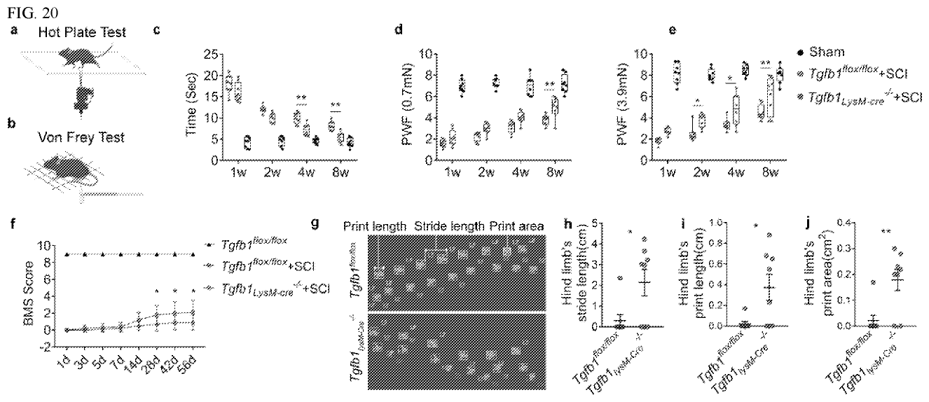

Sensory and motor activity of the SCI mice after conditional knockout of Tgfb1 in macrophage lineage cells was evaluated with the hot plate test (FIG. 20A). Sensory impairments in hindpaw withdrawal time on the hot plate test improved continuously from week 1 to week 8 in SCI Tgfb1_{LysM-cre}^{-/-} mice relative to SCI Tgfb1^{flox/flox} mice (FIG. 20C). Similar recovery results were observed in the manual von Frey test, BMS score of hindlimb function⁴⁰, and gait analysis. Specifically, hindpaw withdrawal frequency on the manual von Frey test increased significantly from 2 weeks after SCI (FIG. 20B, D, E) in SCI Tgfb1LysM-cre^{-/-} mice, and motor impairment improved significantly from 28 days after SCI as assessed by BMS score (FIG. 20F). Finally, gait analysis demonstrated that hindpaw stride length, pawprint length, and pawprint area were significantly increased in SCI Tgfb1_{LysM-cre}^{-/-} mice relative to SCI Tgfb1^{flox/flox} littermates at 8 weeks after SCI (FIG. 20G-J). Taken together, these results suggest that fibrotic scar formation promoted by macrophage-mediated TGF-β activation prohibits functional recovery after SCI.

### Example XIII

This example demonstrates a conditional knockout of TGF-β type 2 receptor (Tgfbr2) in pericytes reduced fibrotic scar formation in SCI mice.

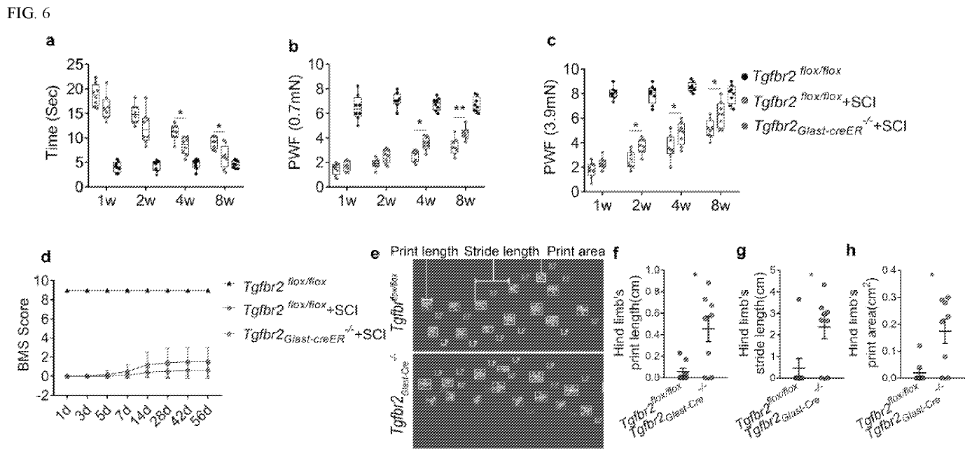

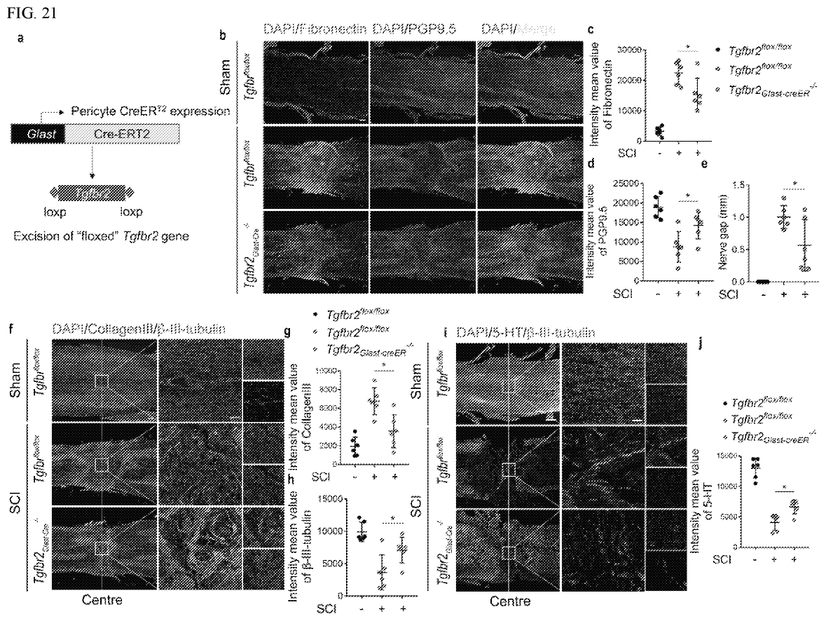

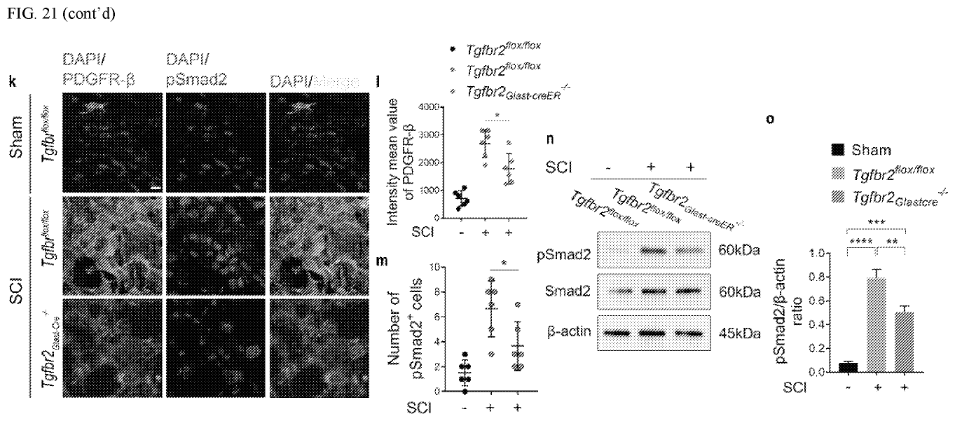

To examine whether pericytes, a subpopulation of MSCs, induced by elevated TGF-β activity form fibrotic scar after SCI, we crossed pericyte-specific Glast-creERT2 mice with Tgfbr2^{flox/flox} mice to knock out Tgfbr2 in pericyte Tgfbr2_{Glast-creER}^{-/-} mice because Tgfbr2 is the only type 2 receptor in TGF-β signaling pathway and has been shown to be involved in fibrosis in different organs^{41,42} (FIG. 21A). Co-immunostaining of PGP9.5 with fibronectin revealed increased neurons and nerve fibers, less fibrotic scar formation, and a narrowed nerve gap in Tgfbr2_{Glast-creER}^{-/-} mice relative to Tgfbr2^{flox/flox} mice (FIG. 21B-E). Similarly, co-immunostaining of collagen III with β-III -tubulin further confirmed decreased fibrotic scar formation and a narrowed nerve gap in Tgfbr2_{Glast-creER}^{-/-} mice (FIG. 21F-H). Moreover, co-immunostaining of 5-HT with β-III-tubulin showed higher levels of serotonin neurotransmitter and more abundant nerve fibers across the site of SCI in Tgfbr2_{Glast-creER}^{-/-} mice compared with Tgfbr2^{flox/flox} mice (FIG. 21I, J), similar to the results in Tgfb1_{LysM-cre}^{-/-} mice. In contrast, as fibrotic scar decreased, glial scar with abundant glial fibrillary acidic protein⁺ (GFAP⁺) astrocytes were significantly increased in Tgfbr2_{Glast-creER}^{-/-} mice (FIG. 9A, B). Importantly, the number of pSmad2⁺PDGFR-β⁺ pericytes decreased significantly in Tgfbr2_{Glast-creER}^{-/-} mice (FIG. 21K-M). Western blot demonstrated that pSmad2 level was significantly increased in SCI Tgfbr2_{Glast-creER}^{-/-} mice relative to sham-operated mice and decreased in SCI Tgfbr2_{Glast-creER}^{-/-} mice (FIG. 21N, O), indicating a large proportion of TGF-β signaling in pericytes. To measure mouse behavior, we used the manual von Frey test, hot plate test, BMS score, and gait analysis of the hindlimb, which showed significant SCI recovery in Tgfbr2_{Glast-creER}^{-/-} mice relative to Tgfbr2^{flox/flox} control mice (FIG. 6). Taken together, these results suggest that TGF-β signaling in pericytes is critical for fibrotic scar formation to prohibit functional recovery in SCI mice.

### Example XIV

This example demonstrates fibroblast differentiation of MSCs secreted axonal growth-inhibitory factors.

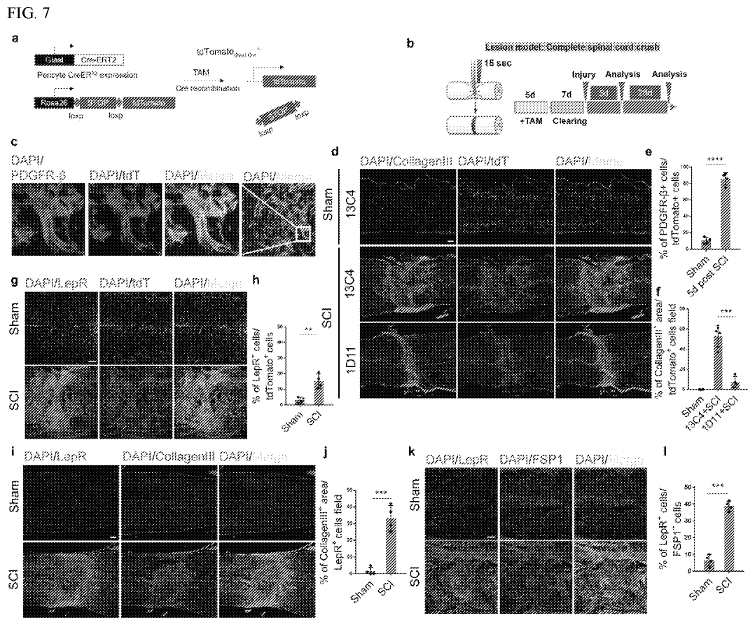

To investigate the types of cells recruited by TGF-β for fibrotic scar formation, we performed a pericyte lineage-tracing experiment using Solute Carrier Family 1 Member 3 (Glast)-CreER^{T2}::ROSA26-tdTomato mice by crossing specific inducible Glast-CreER^{T2} transgenic mice with ROSA26-tdTomato reporter mice, because pericytes in the stromal cell lineage undergo fibroblast differentiation induced by TGF-β. Injection of tamoxifen in Glast-CreER^{T2}::ROSA26-tdTomato mice induced genetically labeling a subpopulation of pericytes and their progeny, potentially forming the fibrotic scar after SCI (FIG. 7A, B).

Immunostaining of PDGFR-β, a marker of pericytes, showed that tdTomato⁺ cells were more than 80% PDGFR-β⁺ cells after SCI, indicating successfully labeling of pericytes with tdTomato (FIG. 7C, E). Importantly, injection of TGF-β neutralizing antibody significantly reduced tdTomato-labeled pericytes in the fibrotic scar relative to injection of 13C4 control antibody, suggesting that pericytes were recruited by TGF-β for fibrotic scar formation (FIG. 7D, F). Moreover, immunostaining of leptin receptor (LepR), a marker for MSCs, showed that 12% of tdTomato⁺ pericytes were LepR⁺ after SCI (FIG. 23G, H). Expression of type III collagen, which was largely co-localized with tdTomato-labeled pericytes at the injury area, was significantly reduced in the TGF-β antibody group relative to the 13C4 antibody control group (FIG. 7I, J), indicating fibrotic differentiation of pericytes. Interestingly, immunostaining of fibroblast-specific protein 1 (FSP1) demonstrated that nearly 40% of FSP1⁺ fibroblasts are LepR⁺ MSCs, indicating that tdTomato-labeled pericytes represent a subpopulation of MSC lineage cells undergoing fibroblast differentiation (FIG. 7K, L).

### Example XV.

This example provides a discussion of Examples VIII-XIV.

Scar formation, including glial and fibrotic scars at SCI sites, was believed the primary obstacle for axon regeneration and functional recovery⁴⁹⁻⁵⁵. Although glial scar has been well studied for its cellular and structural content, and particularly for the potential benefits the formation of glial scar has for functional recovery from SCI⁵⁴⁻⁵⁸, little is known about fibrotic scar. Previous studies of fibrotic scar have been primarily in vitro studies. Fibrotic scar formation is believed, through fibrosis, to inhibit axonal regeneration after SCI⁵⁹⁻⁶¹. We found that overactive TGF-β 13 at SCI sites recruits and induces fibroblast differentiation of MSCs for the formation of fibrotic scar. Importantly, inhibition of TGF-β activity reduced fibrotic scar formation and significantly improved recovery from SCI in mice. Strikingly, neonatal mice did not show TGF-β activity at SCI site and showed a full recovery after injury. Collectively, our findings unraveled that fibrotic scar resulted from abnormal TGF-β activity is the primary obstacle that impedes the recovery after SCI.

In evolution, TGF-βs exist only in mammals and are known to promote tissue repair, remodeling, and homeostasis in adulthood^{62,63}. Unlike most cytokines. TGF-βs are expressed in an inactive latent form and deposited in the matrix upon secretion. After injury or during tissue remodeling, TGF-β are activated to recruit stein cells or progenitors to the injury site where these cells undergo differentiation for tissue repairing and restoring homeostasis²⁶⁻⁶⁷. However, fibrosis in various tissues is often associated with overactive TGF-β^{69,68,69}. Similarly, TGF-β becomes overactive to recruits MSCs for fibroblast differentiation and fibrotic scar formation after SCI^{70,71}.

The nature of the fibrotic scar formation is to repair the tissue injury quickly for survival, However, scar tissues also become a physical and chemical barrier for axon regeneration. This partially explains why the recovery after SCI is particularly difficult in mammals. Apparently, TGF-β activation mechanism has not been developed yet in early neonatal mice, which provides an ideal model to study the role of fibrotic scar formation in SCI recovery.

Fibroblast differentiation of MSCs initiates fibrotic scar formation after SCI. Both TGF-β1 and pSmad2/3 levels were significantly increased after SCI. Conditional knockout of Tgfbr2 in the Glast-pericyte lineage cells significantly decreased fibronectin expression and improved the recovery of SCI mice. Given that pericytes are a subpopulation of MSCs, our results suggest that MSCs were recruited by overactive TGF-β1 for fibrotic scar formation. Single-cell sequencing analysis also revealed that TGF-β receptors, ligands, and their downstream signaling were significantly increased after SCI in 3 days. Moreover, TGF-β signaling pathway profiling of pericytes showed a significant increase, and our lineage trajectory indicates that pericytes differentiate to fibroblasts via MSCs because pericytes possess stem cell characteristics as a subpopulation of MSCs. Since there has been no unique marker for MSCs and Glast expression is relative specific for pericytes, Glast-Cre was used to drive conditional knockout of Tgfbr2 and used in lineage-tracing experiment. Interestingly, the controversial observation of pericyte function in SCI has been reported^{60,72-74}. Our results provide an explanation for these controversial results. Pericytes have potential stem cell capacity and reside within the vascular basement membrane to stabilize the vessels and tight junctions between endothelial cells and to support the structural integrity of the blood-spinal cord barrier. After SCI, TGF-β1 was overactivated to induce migration of pericytes for fibrotic scar formation, which damages the structural integrity of the blood-spinal cord barrier. Exogenous pericyte transplantation has been shown to enhance the structural integrity and function of the new blood vessels, particularly for the regulation of neurovascular function, as a potential therapy for SCI⁷⁵⁻⁷⁷. The transplanted pericytes likely repair blood vessels, compensating for the deficit of pericytes as a result of depletion by overactive TGF-β for fibrotic scar formation after SCI.

In summary, we took multiple approaches to demonstrate that overactive TGF-β recruited MSCs for fibrotic scar formation after SCI. In LysM-cre::iDTR^{flox/flox} mice, macrophage lineage cells were eliminated and TGF-β activity was significantly decreased, suggesting macrophages may be responsible for increased TGF-β1 expression and over activation. TGF-β1 activity was also significantly decreased in LysM-cre: Tgfb1^{flox/flox} mice, which further validated the expression of TGF-β1 in macrophages. Furthermore, findings in Glast-creERT2::Tgfbr2^{flox/flox} mice demonstrate that TGF-β signaling in pericytes drives their fibroblast differentiation. Although the effect was not as strong as that from TGF-β neutralizing antibody 1D11 treatment, all three conditional knockout mice showed significantly improved functional recovery after SCI (FIG. 5H). Neonatal mice with no active TGF-β showed no fibrotic scar formation and near-complete recovery after SCI, further supporting the notion that overactive TGF-β is responsible for fibrotic scar formation, and thus inhibition of abnormal TGF-β over activation indicates a new therapy for SCI.

### Example XVI

This example provides materials and methods related to Examples VIII-XV.

### Genetic Labeling of Transgenic Mice

Recombination in Glast-Rasless-YFP mice was induced by a daily intraperitoneal injection of 2 mg of tamoxifen (20 mg/mL in 1:9 ethanol:corn oil, Sigma-Aldrich, St. Louis, MO, USA) for 5 consecutive days (FIG. 17B). Vehicle mice (matched mice with the same genotype) received the same number of injections of the solvent (1:9 ethanol:corn oil) without tamoxifen. Mice were randomly assigned to the vehicle or tamoxifen group. Glast-Tdtomato mice were recombined with tamoxifen using the aforementioned protocol. Injuries were performed after a 7-day clearing period starting after the last tamoxifen injection. Tamoxifen and its active metabolite 4-hydroxytamoxifen have a half-life of 6-12 hours in the mouse^{73,78}. A previous study analyzing CreERT2 distribution in the adult mouse spinal cord 6 days after the last tamoxifen administration showed that there is no CreERT2 in the nucleus of cells at this time, directly demonstrating that tamoxifen has been cleared at this time point⁷⁹. Therefore, the chosen 7-day washout period ensures that no tamoxifen is left at the time of injury or after, which could affect the response to injury. Moreover, the 7-day washout period guarantees that all recombination occurs before the injury; therefore, if cells other than type A pericytes start expressing the Glast-CreERT2 transgene in response to the injury, recombination will not occur⁷³.

### Mice

All young adult C57BL/6J male and female mice were 8 weeks of age at the time of SCI. All mice were housed in a 12-hour light/dark cycle in a specific pathogen-free facility with controlled temperature and humidity and were allowed free access to food and water. Animal care, including manual bladder voiding, was performed at least twice daily or as needed after SCI for the duration of the experiment.

We purchased the 8-week-old LysM-cre (Stock number: 004781), iDTR^{flox/flox} (Stock number: 007900), Tgfb1^{flox/flox} (Stock number: 010721) and Glast-creERT2 (Stock number: 012586) mouse strains from the Jackson Laboratory (Bar Harbor, ME, USA). Tgfbr2^{flox/flox} were obtained from the laboratory of H. L. Moses (Vanderbilt University, Nashville, Tennessee, USA)⁸⁰ . We generated LysM-cre::iDTR^{flox,flox} mice (iDTR_{LysM-cre}^{-/-}) by crossing LysM-cre mice with iDTR^{flox/flox} mice, LysM-cre::Tgfb1^{flox/flox} mice (Tgfb1_{LysM-cre}^{-/-}) by crossing LysM-cre mice with Tgfb1^{flox/flox} mice and Glast-creERT2::Tgfbr2^{flox/flox} mice (Tgfbr2_{Glast-creER}^{-/-}) by crossing Glast-creERT2 mice with Tgfbr2^{flbr}2^{flox/flox} mice. We induced SCI in 8-week-old WT, iDTR_{LysM-cre}^{-/-}, iDTR^{flox/flox}, Tgfb1_{LysM-cre}^{-/-}, Tgfb1^{flox/flox}, Tgfbr2_{Glast-creER}^{-/-}, and Tgfbr2^{flox/flox} fox female mice and humanely killed them at different time points.

For the antibody treatment experiments, we purchased 8-week-old C57BL/6J (WT, Stock number: 000664) mice from the Jackson Laboratory, and they were intraperitoneally injected with 13C4 (R&D Systems, Minneapolis, MN) or 1D11 (R&D Systems), 5 mg/kg body weight, 3 times a week for 1, 2, or 4 weeks after SCI (FIG. 17B). 1D11 is a monoclonal antibody that neutralizes 3 major active TGF-β isoforms (TGF-β1, −2, and −3), the known ligands for the TGF-β receptor kinase.

We purchased 8-week-old R26R-TdTomato mice (Stock number: 007909) from the Jackson Laboratory (Bar Harbor, ME, USA). We generated Glast-creERT2::R26R-TdTomato mice by crossing Glast-creERT2 mice with R26R-TdTomato mice. We performed SCI operations on 8-week-old Nestin-creERT2::R26R-TdTomato male mice. Three days after surgery, we treated the mice with 80 mg/kg body weight of tamoxifen 3 times a week for 2 or 4 weeks and humanely killed the mice at 2 or 4 weeks after surgery.

All mice were maintained in the animal facility of The Johns Hopkins University School of Medicine (Baltimore, MD, USA). The experimental protocols were reviewed and approved by the Institutional Animal Care and Use Committee of The Johns Hopkins University.

### Surgical Procedures

We anesthetized the mice at 8 weeks of age with ketamine (Vetalar, Ketaset, Ketalar; 100 mg/kg, intraperitoneally) and xylazine (Rompun, Sedazine, Anased; 10 mg/kg, intraperitoneally). We then shaved the hair ranging from 2-3 cm above and below the T10 position of the back (the highest raised point on the back) and disinfected the operating table and surgical instruments in advance, laying towels to prepare the instruments. After a 1-cm skin incision was made at the T10 position on the back, the muscles in the local area were bluntly separated, using gauze to stop bleeding, and then the T10 lamina was exposed. Subsequently, severe crush SCIs were made at the level of T10 after laminectomy of a single vertebra by using Dumont #5 Forceps (Fine Science Tools, Foster City, CA, USA) without spacers and with a tip width of 0.5 mm to completely compress the entire spinal cord laterally from both sides for 15 seconds^{7,53,81,82}. Spastic tail swings, retraction, and fluttering of the lower extremities and body, paralysis of both lower extremities, and dura mater congestion and were observed, indicating successful modeling (FIG. 10A). Finally, the wound was washed with normal saline. After hemostasis with gelatin sponge, the muscle and skin were sutured layer by layer. Warm blankets were used to rewarm mice postoperatively to prevent hypothermia. Mice were housed in cages, and manual assistance was provided to mice at least twice a day to empty the bladder to avoid urinary system infections. The sham group does not perform spinal cord compression and the other operations are the same as the above. Neonatal (P2/P7/P12) mice were anesthetized using isoflurane. A laminectomy was performed at the thoracic level (T10) until the spinal cord was exposed completely from side-to-side. The spinal cord was then fully crushed for 5 seconds. After SCI, the pups were returned to the mother. Feeding was monitored closely in the first week after surgery. Nutra-Gel diets (Bio-Serv, Flemington, NJ, USA) or breeder chow diets were provided to avoid cannibalism.

### ELISA (Enzyme-Linked Immunosorbent Assay) and Western blot.

We determined the concentration of active and total TGF-β1 in the conditioned media using the ELISA Development Kit (R&D Systems, MB100B) according to the manufacturer's instructions. Western blot analyses were conducted on the protein of lysates from the in vivo spinal cord. The spinal cord lysates were centrifuged, and the supernatants were separated by SDS-PAGE (sodium dodecyl sulfate-polyacrylamide gel electrophoresis) and blotted on polyvinylidene fluoride membrane (Bio-Rad Laboratories, Hercules, CA, USA). After incubation in specific antibodies, we detected proteins using an enhanced chemiluminescence kit (Amersham Biosciences, Little Chalfont, UK). We used antibodies recognizing mouse pSmad2 (1:1000, Cell Signaling Technology, Inc., Danvers, MA, USA) and Smad2 (1:1000, Cell Signaling Technology, Inc.) to examine the protein concentrations in the lysates.

### Histochemistry and Immunohistochemistry

At the time of euthanasia, the spinal cords were collected and fixed in 4% paraformaldehyde overnight and then embedded in paraffin or optimal cutting temperature compound after being dehydrated with 30% sucrose for 48 hours (Sakura Finetek, Torrance, CA, USA). Four-μm-thick, coronal-oriented sections of the spinal cord were processed for immunohistochemistry staining using a standard protocol. Thirtyμm-thick, coronal-oriented sections were prepared for nerve-related immunofluorescent staining, and 10-μm-thick, coronal-oriented sections were used for scar-related and other immunofluorescent staining using a standard protocol. The sections were incubated with primary antibodies to rabbit 5-HT (1:50, sc-65495, Santa Cruz Biotechnology, Dallas, TX, USA), mouse β-III-tubulin (1:100, MA1-118, Invitrogen, Carlsbad, CA, USA), rabbit PGP9.5 (1:250, ab108986, Abcam, Cambridge, UK), mouse PGP9.5 (1:50, ab8189, Abcam), rabbit Fibronectin (1:100, ab2413, Abcam), mouse Fibronectin (1:100, ab6328, Abcam), rabbit Collagen III (1:100, ab7778, Abcam), chicken GFAP (1:500, ab4674, Abcam), mouse Collagen 1α1 (1:50, sc-293182, Santa Cruz Biotechnology), rabbit Phospho SAMD2 (1:100, 44-244G, Invitrogen), rabbit PDGFR-β3 (1:100, ab32570, Abcam), goat PDGFR-β (1:100, AF1042, R&D Systems), rabbit FSP1 (1:300, 07-2274, MilliporeSigma, Burlington, MA, USA), and chicken green fluorescent protein (1:250, ab13970, Abcam) overnight at 4° C. Then, the corresponding secondary antibodies were added onto the sections for 1 hour while avoiding light. For immunohistochemistry, a horseradish peroxidase-streptavidin detection system (Dako, Carpinteria, CA, USA) was subsequently used to detect the immunoactivity, followed by counterstaining with hematoxylin (Sigma-Aldrich). For immunofluorescent staining, the sections were counterstained with 4′,6-diamidino-2-phenylindole (DAPI) (H-1200, Vector Laboratories, Burlingame, CA, USA). The sample images were observed and captured by a fluorescence microscope (BX51, DP71, Olympus Scientific Solutions Americas Inc., Waltham, MA, USA) or confocal microscope (LSM 780, Zeiss, Oberkochen, Germany). ImageJ software (National Institutes of Health, Bethesda, MD, USA) was used for quantitative analysis. We calculated nerve and scar area as described in a previous study⁸³.

### Quantification Analysis

Five alternate sagittal sections per mouse, spanning the lesion center and spaced 130 mm apart, were immunostained for 5-HT, β-III -tubulin, PGP9.5, fibronectin, collagen III, pSmad2, PDGFR-β, Ephb2, Sema3A, GFAP, and DAPI. The lesion site was photographed using a Zeiss LSM 780 confocal microscope or Olympus BX51 microscope, and the lesion center was manually outlined. Measurements were performed using ImageJ/Fiji software. The nerve gap, which was determined using ImageJ/Fiji, was the distance between the rostral and caudal ends of PGP9.5⁺ nerve in the injury area. The fibrotic scar area occupied by collagen III signal was thresholded and determined by ImageJ/Fiji software.

### Behavioral Testing

Behavioral testing was performed before surgery and weekly after surgery. All behavioral tests were performed by the same investigator, who was blinded to the study groups.

The hindpaw withdrawal frequency in response to a mechanical stimulus was determined using von Frey filaments of 0.7 mN and 3.9 mN (Stoelting Co., Wood Dale, IL, USA). Mice were placed on a metal wire mesh grid covered with a clear plastic cage. Mice were allowed to acclimatize to the environment for 30 minutes before testing. Von Frey filaments were applied to the mid-plantar surface of the hindpaw through the mesh floor with enough pressure to buckle the filaments. Probing was performed only when the mouse's paw was in contact with the floor. A trial consisted of application of a von Frey filament to the hindpaw 10 times at 1-second intervals. If withdrawal occurred after application, it was recorded, and the next application was performed similarly when the mouse's paw was again in contact with the floor. Mechanical withdrawal frequency was calculated as the percentage of withdrawals in response to 10 applications.

We performed automated gait analysis preoperatively and 1, 2, 4, and 8 weeks postoperatively using a “CatWalk” system (Noldus, Leesburg, VA, USA). All experiments were performed during the same period of the day (1:00 PM to 4:00 PM) and analyzed as previously reported^{84,85}. Briefly, we trained mice to cross the CatWalk walkway daily for 7 days before SCI or control operation. During the test, each mouse was placed individually in the CatWalk walkway, which consists of a glass plate (100×15×0.6 cm) plus two Plexiglas walls, spaced 8 cm apart. The mice were allowed to walk freely and traverse from one side of the walkway glass plate to the other. Two infrared light beams spaced 90 cm apart were used to detect the arrival of the mouse and to control the start and end of data acquisition. We recorded these data when the room was completely dark, with the exception of the light from the computer screen. LED light from an encased fluorescent lamp was emitted inside the glass plate and completely internally reflected. When the mouse paws made contact with the glass plate, light was reflected down and the illuminated contact area was recorded with a high-speed color video camera positioned under the glass plate and connected to a computer running CatWalk software, version 9.1 (Noldus). We compared stride length, print length, and print area in each run of each mouse at each time point. Paired t-tests were used for statistical analysis.

The hot plate test was used to calculate analgesic activity using the method described by Eddy and Leimbach⁸⁶ with minor modifications. Mice were retained on a hot plate having a stable temperature of 42° C. Each mouse was placed individually on the hot plate to observe their reaction to electrical heat-induced pain (licking of the forepaws and eventually jumping). The time taken for either paw licking or jumping was recorded. The latency until mice showed the first signs of discomfort (hindpaw lifting, hindpaw licking, or jumping) was recorded, and responses were determined at 1, 2, and 4 weeks after SCI.

Mice were also tested for hindlimb functional deficits at 1, 3, and 5 days and 1, 2, 4, 6, and 8 weeks (n=6 per group) after SCI. Hindlimb locomotor recovery was assessed in an open field using the BMS score⁴⁰. This scale ranges from 0 (indicating complete paralysis) to 9 (indicating normal movement of the hindlimbs). Performance of the left and right hindlimbs was averaged to obtain the BMS score.

### Single-Cell RNA-Seq (scRNAseq) Analysis

Single-cell suspension was prepared using the 10X Genomics Chromium Single Cell 3′ Reagent Kit v3 (10X Genomics) according to the manufacturer's protocol. The quantity and quality of cDNA were assessed using an Agilent 2100 Expert High Sensitivity DNA Assay. cDNA samples were sequenced on one lane of a NovaSeq 6000 S2 flowcell at Johns Hopkins School of Medicine for Genomics and Bioinformatics. Sequence alignment to the GRCm38 (mm10) reference genome was performed using Cell Ranger v.5.0.1⁸⁷. More than 430 million reads were obtained for each sample. The average number of genes detected per cell was 848±103 (mean±s.e.m). Subsequent quality control filtering, normalization, clustering, and differential gene expression analysis was performed using Seurat (v.4.0.0, https://github.com/satijalab/seurat)⁸⁸. Quality control, normalization, log transformation, and highly variable gene identification were performed separately for each condition (sham, 13C4, 1D11). For each dataset, genes expressed in fewer than five cells were removed and ribosomal, mitochondrial, and Metastasis Associated Lung Adenocarcinoma Transcript 1 (MALAT1) genes were removed. Doublets and debris were removed by selecting for cells with mitochondrial gene content less than 25% of their total reads, a minimum of 200 unique features, and a maximum number of unique features set as the 95^{th} percentile of the number of features detected per cell. The datasets were each normalized, log transformed, and highly variable genes were identified. After quality control filtering, the datasets contained 233 cells (1D11 condition), 281 cells (13C4 condition), and 238 cells (Sham condition), respectively.

### Batch Correction, Clustering, and Cell Type Annotation.

The datasets for the three conditions (1D11, 13C4, and Sham) were merged in Seurat, and highly variable genes for the merged data were found to be the union of highly variable gene (HVG) for each individual sample and for the merged sample (total 4122 genes). The merged dataset was scaled, principal component analysis (PCA) was performed, and scores for expression of G2M and S phase cell cycle markers were computed and visualized on principal components (PC) coordinates. Batch correction was performed using Harmony (version 0.1.0, https://github.com/immunogenomics/harmony)⁸⁹ to re-compute corrected embeddings. The Leiden algorithm was used to perform clustering on the corrected embeddings and clusters were visualized on a uniform manifold approximation and projection (UMAP) embedding. Cluster annotation was performed by visualizing key marker gene expression on a UMAP embedding, performing differential expression analysis using DESeq2 (version 1.30.1)⁹⁰, and performing classification using the Python version of SingleCellNet (https://github.com/pcahan1/PySingleCellNet)⁹¹ using a Random Forest classifier trained on Tabula Muris Senis 10X data from skeletal muscle⁹².

### Downstream Analyses: Pseudotime and Ligand-Receptor Interactions

Pseudotime was computed on Harmony embeddings using Slingshot (version 1.8.0, https://github.com/kstreet13/slingshot)⁹³. The change in gene expression over pseudotime of key TGF-β pathway genes was visualized in a heat map. Ligand-receptor interactions were interrogated using CellPhoneDB (version 2.0, https://github.com/Teichlab/cellphonedb)⁹⁴, a repository of receptor-ligand interactions which accounts for subunit architecture and method to infer cell-cell communication networks from scRNAseq data. CellPhoneDB's dot plot function was used to visualize significant ligand-receptor interactions (p<0.05) between cell types of interest. TGF-β target enrichment was performed using enrichR (https://github.com/wjawaid/enrichR)^{95,96} to measure the extent to which genes more highly expressed in 13C4 overlapped with TGF-β effector target genes, as determined by Chromatin immunoprecipitation (ChIP)-Seq data of Smad1, Smad2, Smad3, and Smad4, as compiled in Epoch (https://github.com/pcahan1/epoch).

### Statistics

Data are presented as means±standard deviations. The comparisons for Osteoarthritis Research Society International (OARSI) scores, bone mass, and microarchitecture among different groups were performed using multifactorial analysis of variance (ANOVA). When ANOVA testing indicated overall significance of main effects without interaction between them, the difference between individual time points and sites was assessed by post hoc tests. The level of significance was set at P<0.05. All data analyses were performed using SPSS Statistics analysis software, version 15.0 (SPSS Inc, IBM Corp., Armonk, NY, USA).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. In particular, the following references are denoted within the specification and are herein incorporated by reference in their entireties:

1 McDonald, J. W. & Sadowsky, C. Spinal-cord injury. Lancet 359, 417-425, doi:10.1016/S0140-6736(02)07603-1 (2002).

2 Holmes, D. Spinal-cord injury: spurring regrowth. Nature 552, S49, doi:10.1038/d41586-017-07550-9 (2017).

3 Spinal cord injury facts and figures at a glance. J Spinal Cord Med 37, 117-118, doi:10.1179/1079026813Z.000000000249 (2014).

4 Thuret, S., Moon, L. D. & Gage, F. H. Therapeutic interventions after spinal cord injury. Nat Rev Neurosci 7, 628-643, doi:10.1038/nm1955 (2006).

5 Zhu, Y. et al. Hematogenous macrophage depletion reduces the fibrotic scar and increases axonal growth after spinal cord injury. Neurobiol Dis 74, 114-125, doi:10.1016/j.nbd.2014.10.024 (2015).

6 Mothe, A. J. & Tator, C. H. Advances in stem cell therapy for spinal cord injury. J Clin Invest 122, 3824-3834, doi:10.1172/JCI64124 (2012).

7 Anderson, M. A. et al. Astrocyte scar formation aids central nervous system axon regeneration. Nature 532, 195-200, doi:10.1038/nature17623 (2016).

8 Dias, D. 0. & Goritz, C. Fibrotic scarring following lesions to the central nervous system. Matrix Biol 68-69, 561-570, doi:10.1016/j.matbio.2018.02.009 (2018).

9 Courtine, G. & Sofroniew, M. V. Spinal cord repair: advances in biology and technology. Nat Med 25, 898-908, doi:10.1038/s41591-019-0475-6 (2019).

10 Tang, X., Davies, J. E. & Davies, S. J. Changes in distribution, cell associations, and protein expression levels of NG2, neurocan, phosphacan, brevican, versican V2, and tenascin-C during acute to chronic maturation of spinal cord scar tissue. J Neurosci Res 71, 427-444, doi:10.1002/jnr.10523 (2003).

11 Braga, T. T., Agudelo, J. S. & Camara, N. O. Macrophages During the Fibrotic Process: M2 as Friend and Foe. Front Immunol 6, 602, doi:10.3389/fimmu.2015.00602 (2015).

12 Perrin, F. E., Lacroix, S., Aviles-Trigueros, M. & David, S. Involvement of monocyte chemoattractant protein-1, macrophage inflammatory protein-1alpha and interleukin-1beta in Wallerian degeneration. Brain 128, 854-866, doi:10.1093/brain/awh407 (2005).

13 Kigerl, K. A. et al. Identification of two distinct macrophage subsets with divergent effects causing either neurotoxicity or regeneration in the injured mouse spinal cord. J Neurosci 29, 13435-13444, doi:10.1523/JNEUROSCI.3257-09.2009 (2009).

14 Zhou, X. et al. Microglia and macrophages promote corralling, wound compaction and recovery after spinal cord injury via Plexin-B2. Nat Neurosci 23, 337-350, doi:10.1038/s41593-020-0597-7 (2020).

15 Lin, S. L., Kisseleva, T., Brenner, D. A. & Duffield, J. S. Pericytes and perivascular fibroblasts are the primary source of collagen-producing cells in obstructive fibrosis of the kidney. Am J Pathol 173, 1617-1627, doi:10.2353/ajpath.2008.080433 (2008).

16 Hines, D. J., Hines, R. M., Mulligan, S. J. & Macvicar, B. A. Microglia processes block the spread of damage in the brain and require functional chloride channels. Glia 57, 1610-1618, doi:10.1002/glia.20874 (2009).

17 Hara, M. et al. Interaction of reactive astrocytes with type I collagen induces astrocytic scar formation through the integrin-N-cadherin pathway after spinal cord injury. Nat Med 23, 818-828, doi:10.1038/nm.4354 (2017).

18 Xu, B. et al. Role of CSPG receptor LAR phosphatase in restricting axon regeneration after CNS injury. Neurobiol Dis 73, 36-48, doi:10.1016/j.nbd.2014.08.030 (2015).

19 Goritz, C. et al. A pericyte origin of spinal cord scar tissue. Science 333, 238-242, doi:10.1126/science.1203165 (2011).

20 Hynes, R. O. The extracellular matrix: not just pretty fibrils. Science 326, 1216-1219, doi:10.1126/science.1176009 (2009).

21 Distler, J. H. W. et al. Shared and distinct mechanisms of fibrosis. Nat Rev Rheumatol 705-730, doi:10.1038/s41584-019-0322-7 (2019).

22 Leask, A. Targeting the TGFbeta, endothelin-1 and CCN2 axis to combat fibrosis in scleroderma. Cell Signal 20, 1409-1414, doi:10.1016/j.cellsig.2008.01.006 (2008).

23 Wilson, S. E., Marino, G. K., Torricelli, A. A. M. & Medeiros, C. S. Injury and defective regeneration of the epithelial basement membrane in corneal fibrosis: A paradigm for fibrosis in other organs? Matrix Biol 64, 17-26, doi:10.1016/j.matbio.2017.06.003 (2017).

24 Tang, Y. et al. TGF-beta1-induced migration of bone mesenchymal stem cells couples bone resorption with formation. Nat Med 15, 757-765, doi:10.1038/nm.1979 (2009). Wan, M. et al. Injury-activated transforming growth factor beta controls mobilization of mesenchymal stem cells for tissue remodeling. Stem Cells 30, 2498-2511, doi:10.1002/stem.1208 (2012).

26 Wang, X. et al. Inhibition of overactive TGF-beta attenuates progression of heterotopic ossification in mice. Nat Commun 9, 551, doi:10.1038/s41467-018-02988-5 (2018).

27 Zhen, G. et al. Inhibition of TGF-beta signaling in mesenchymal stem cells of subchondral bone attenuates osteoarthritis. Nat Med 19, 704-712, doi:10.1038/nm.3143 (2013).

28 Zhen, G. et al. Mechanical stress determines the configuration of TGFbeta activation in articular cartilage. Nat Commun 12, 1706, doi:10.1038/s41467-021-21948-0 (2021).

29 Flanders, K. C., Ren, R. F. & Lippa, C. F. Transforming growth factor-betas in neurodegenerative disease. Prog Neurobiol 54, 71-85, doi:10.1016/s0301-0082(97)00066-x (1998).

30 Di Gregorio, J. et al. The Epithelial-to-Mesenchymal Transition as a Possible Therapeutic Target in Fibrotic Disorders. Front Cell Dev Biol 8, 607483, doi:10.3389/fcell.2020.607483 (2020).

31 El Agha, E. et al. Mesenchymal Stem Cells in Fibrotic Disease. Cell Stem Cell 21, 166-177, doi:10.1016/j.stem.2017.07.011 (2017).

32 Usunier, B., Benderitter, M., Tamarat, R. & Chapel, A. Management of fibrosis: the mesenchymal stromal cells breakthrough. Stem Cells Int 2014, 340257, doi:10.1155/2014/340257 (2014).

33 Li, Y. et al. Microglia-organized scar-free spinal cord repair in neonatal mice. Nature 587, 613-618, doi:10.1038/s41586-020-2795-6 (2020).

34 Inman, D., Guth, L. & Steward, O. Genetic influences on secondary degeneration and wound healing following spinal cord injury in various strains of mice. J Comp Neurol 451, 225-235, doi:10.1002/cne.10340 (2002).

35. Antri, M., Orsal, D. & Barthe, J. Y. Locomotor recovery in the chronic spinal rat: effects of long-term treatment with a 5-HT2 agonist. Eur J Neurosci 16, 467-476, doi:10.1046/j.1460-9568.2002.02088.x (2002).

36 Musienko, P. et al. Controlling specific locomotor behaviors through multidimensional monoaminergic modulation of spinal circuitries. J Neurosci 31, 9264-9278, doi:10.1523/JNEUROSCI.5796-10.2011 (2011).

37 Slawinska, U., Miazga, K. & Jordan, L. M. The role of serotonin in the control of locomotor movements and strategies for restoring locomotion after spinal cord injury. Acta Neurobiol Exp (Wars) 74, 172-187 (2014).

38 Ahn, M. J. & Cho, G. W. Metformin promotes neuronal differentiation and neurite outgrowth through AMPK activation in human bone marrow-mesenchymal stem cells. Biotechnol Appl Biochem 64, 836-842, doi:10.1002/bab.1584 (2017).

39 Hu, S. et al. Substantial Neuroprotective and Neurite Outgrowth-Promoting Activities by Bis(propyl)-cognitin via the Activation of Alpha7-nAChR, a Promising Anti-Alzheimer's Dimer. ACS Chem Neurosci 6, 1536-1545, doi:10.1021/acschemneuro.5b00108 (2015).

40 Basso, D. M. et al. Basso Mouse Scale for locomotion detects differences in recovery after spinal cord injury in five common mouse strains. J Neurotrauma 23, 635-659, doi:10.1089/neu.2006.23.635 (2006).

41 Pohlers, D. et al. TGF-beta and fibrosis in different organs - molecular pathway imprints. Biochim Biophys Acta 1792, 746-756, doi:10.1016/j.bbadis.2009.06.004 (2009).

42 Parichatikanond, W., Luangmonkong, T., Mangmool, S. & Kurose, H. Therapeutic Targets for the Treatment of Cardiac Fibrosis and Cancer: Focusing on TGF-beta Signaling. Front Cardiovasc Med 7, 34, doi:10.3389/fcvm.2020.00034 (2020).

43 Garcia, J. et al. Tiel deficiency induces endothelial-mesenchymal transition. EMBO Rep 13, 431-439, doi:10.1038/embor.2012.29 (2012).

44 Pinto, M. T., Covas, D. T., Kashima, S. & Rodrigues, C. O. Endothelial Mesenchymal

Transition: Comparative Analysis of Different Induction Methods. Biol Proced Online 18, 10, doi:10.1186/s12575-016-0040-3 (2016).

45 Bansal, R., van Baarlen, J., Storm, G. & Prakash, J. The interplay of the Notch signaling in hepatic stellate cells and macrophages determines the fate of liver fibrogenesis. Sci Rep 5, 18272, doi:10.1038/srep18272 (2015).

46 Bloom, O. Non-mammalian model systems for studying neuro-immune interactions after spinal cord injury. Exp Neurol 258, 130-140, doi:10.1016/j.expneurol.2013.12.023 (2014).

47 Lee-Liu, D., Edwards-Faret, G., Tapia, V. S. & Larrain, J. Spinal cord regeneration: lessons for mammals from non-mammalian vertebrates. Genesis 51, 529-544, doi:10.1002/dvg.22406 (2013).

48 Zhang, Z., Li, F. & Sun, T. Does repair of spinal cord injury follow the evolutionary theory? Neural Regen Res 7, 849-852, doi:10.3969/j.issn.1673-5374.2012.11.009 (2012).

49 Fernandez-Klett, F. & Priller, J. The fibrotic scar in neurological disorders. Brain Pathol 24, 404-413, doi:10.1111/bpa.12162 (2014).

50 Hellal, F. et al. Microtubule stabilization reduces scarring and causes axon regeneration after spinal cord injury. Science 331, 928-931, doi:10.1126/science.1201148 (2011).

51 Li, Y. et al. RNAi-mediated ephrin-B2 silencing attenuates astroglial-fibrotic scar formation and improves spinal cord axon growth. CNS Neurosci Ther 23, 779-789, doi:10.1111/cns.12723 (2017).

52 Tran, A. P., Warren, P. M. & Silver, J. The Biology of Regeneration Failure and

Success After Spinal Cord Injury. Physiol Rev 98, 881-917, doi:10.1152/physrev.00017.2017 (2018).

53 Wanner, I. B. et al. Glial scar borders are formed by newly proliferated, elongated astrocytes that interact to corral inflammatory and fibrotic cells via STAT3-dependent mechanisms after spinal cord injury. J Neurosci 33, 12870-12886, doi:10.1523/JNEUROSCI.2121-13.2013 (2013).

54 Alizadeh, A. et al. Neuregulin-1 positively modulates glial response and improves neurological recovery following traumatic spinal cord injury. Glia 65, 1152-1175, doi:10.1002/glia.23150 (2017).

55 Li, L., Ni, L., Eugenin, E. A., Heary, R. F. & Elkabes, S. Toll-like receptor 9 antagonism modulates astrocyte function and preserves proximal axons following spinal cord injury. Brain Behav Immun 80, 328-343, doi:10.1016/j.bbi.2019.04.010 (2019).

56 Rolls, A., Shechter, R. & Schwartz, M. The bright side of the glial scar in CNS repair. Nat Rev Neurosci 10, 235-241, doi:10.1038/nm2591 (2009).

57 Seo, T. B., Chang, I. A., Lee, J. H. & Namgung, U. Beneficial function of cell division cycle 2 activity in astrocytes on axonal regeneration after spinal cord injury. J Neurotrauma 30, 1053-1061, doi:10.1089/neu.2012.2693 (2013).

58 White, R. E. et al. Transforming growth factor alpha transforms astrocytes to a growth-supportive phenotype after spinal cord injury. J Neurosci 31, 15173-15187, doi:10.1523/JNEUROSCI.3441-11.2011 (2011).

59 Cooper, J. G. et al. Fibronectin EDA forms the chronic fibrotic scar after contusive spinal cord injury. Neurobiol Dis 116, 60-68, doi:10.1016/j.nbd.2018.04.014 (2018). Soderblom, C. et al. Perivascular fibroblasts form the fibrotic scar after contusive spinal cord injury. J Neurosci 33, 13882-13887, doi:10.1523/JNEUROSCI.2524-13.2013 (2013).

61 Zhou, T. et al. Microvascular endothelial cells engulf myelin debris and promote macrophage recruitment and fibrosis after neural injury. Nat Neurosci 22, 421-435, doi:10.1038/s41593-018-0324-9 (2019).

62 Voisin, A. et al. Differential expression and localisation of TGF-beta isoforms and receptors in the murine epididymis. Sci Rep 10, 995, doi:10.1038/s41598-020-57839-(2020).

63 Xu, X. et al. Transforming growth factor-beta in stem cells and tissue homeostasis. Bone Res 6, 2, doi:10.1038/s41413-017-0005-4 (2018).

64 Crane, J. L. & Cao, X. Bone marrow mesenchymal stem cells and TGF-beta signaling in bone remodeling. J Clin Invest 124, 466-472, doi:10.1172/JCI70050 (2014).

65 Hinz, B. The extracellular matrix and transforming growth factor-betal: Tale of a strained relationship. Matrix Biol 47, 54-65, doi:10.1016/j.matbio.2015.05.006 (2015).

66 Kubiczkova, L., Sedlarikova, L., Hajek, R. & Sevcikova, S. TGF-beta - an excellent servant but a bad master. J Transl Med 10, 183, doi:10.1186/1479-5876-10-183 (2012).

67 MacFarlane, E. G., Haupt, J., Dietz, H. C. & Shore, E. M. TGF-beta Family Signaling in Connective Tissue and Skeletal Diseases. Cold Spring Harb Perspect Biol 9, doi:10.1101/cshperspect.a022269 (2017).

68 Blobe, G. C., Schiemann, W. P. & Lodish, H. F. Role of transforming growth factor beta in human disease. N Engl J Med 342, 1350-1358, doi:10.1056/NEJM200005043421807 (2000).

69 Kim, K. K., Sheppard, D. & Chapman, H. A. TGF-beta1 Signaling and Tissue Fibrosis. Cold Spring Harb Perspect Biol 10, doi:10.1101/cshperspect.a022293 (2018).

70 Fang, S. et al. Umbilical Cord-Derived Mesenchymal Stem Cell-Derived Exosomal MicroRNAs Suppress Myofibroblast Differentiation by Inhibiting the Transforming Growth Factor-beta/SMAD2 Pathway During Wound Healing. Stem Cells Transl Med 5, 1425-1439, doi:10.5966/sctm.2015-0367 (2016).

71 Shi, L. et al. Extracellular vesicles derived from umbilical cord mesenchymal stromal cells alleviate pulmonary fibrosis by means of transforming growth factor-beta signaling inhibition. Stem Cell Res Ther 12, 230, doi:10.1186/s13287-021-02296-8 (2021).

72 Armulik, A. et al. Pericytes regulate the blood-brain barrier. Nature 468, 557-561, doi:10.1038/nature09522 (2010).

73 Dias, D. O. et al. Reducing Pericyte-Derived Scarring Promotes Recovery after Spinal Cord Injury. Cell 173, 153-165 e122, doi:10.1016/j.ce11.2018.02.004 (2018).

74 Li, Y. et al. Pericytes impair capillary blood flow and motor function after chronic spinal cord injury. Nat Med 23, 733-741, doi:10.1038/nm.4331 (2017).

75 Cathery, W., Faulkner, A., Maselli, D. & Madeddu, P. Concise Review: The Regenerative Journey of Pericytes Toward Clinical Translation. Stem Cells 36, 1295-1310, doi:10.1002/stem.2846 (2018).

76 Laredo, F., Plebanski, J. & Tedeschi, A. Pericytes: Problems and Promises for CNS Repair. Front Cell Neurosci 13, 546, doi:10.3389/fncel.2019.00546 (2019).

77 Lu, Y. et al. Bone Mesenchymal Stem Cell-Derived Extracellular Vesicles Promote Recovery Following Spinal Cord Injury via Improvement of the Integrity of the Blood-Spinal Cord Barrier. Front Neurosci 13, 209, doi:10.3389/fnins.2019.00209 (2019).

78 Robinson, S. P., Langan-Fahey, S. M., Johnson, D. A. & Jordan, V. C. Metabolites, pharmacodynamics, and pharmacokinetics of tamoxifen in rats and mice compared to the breast cancer patient. Drug Metab Dispos 19, 36-43 (1991).

79 Meletis, K. et al. Spinal cord injury reveals multilineage differentiation of ependymal cells. PLoS Biol 6, e182, doi:10.1371/journal.pbio.0060182 (2008).

80 Chytil, A., Magnuson, M. A., Wright, C. V. & Moses, H. L. Conditional inactivation of the TGF-beta type II receptor using Cre:Lox. Genesis 32, 73-75, doi:10.1002/gene.10046 (2002).

81 Faulkner, J. R. et al. Reactive astrocytes protect tissue and preserve function after spinal cord injury. J Neurosci 24, 2143-2155, doi:10.1523/JNEUROSCI.3547-03.2004 (2004).

82 Herrmann, J. E. et al. STAT3 is a critical regulator of astrogliosis and scar formation after spinal cord injury. J Neurosci 28, 7231-7243, doi:10.1523/JNEUROSCI.1709-08.2008 (2008).

83 Anderson, M. A. et al. Required growth facilitators propel axon regeneration across complete spinal cord injury. Nature 561, 396-400, doi:10.1038/s41586-018-0467-6 (2018).

84 Angeby-Moller, K., Berge, O. G. & Hamers, F. P. Using the CatWalk method to assess weight-bearing and pain behaviour in walking rats with ankle joint monoarthritis induced by carrageenan: effects of morphine and rofecoxib. J Neurosci Methods 174, 1-9, doi:10.1016/j.jneumeth.2008.06.017 (2008).

85 Hamers, F. P., Koopmans, G. C. & Joosten, E. A. CatWalk-assisted gait analysis in the assessment of spinal cord injury. J Neurotrauma 23, 537-548, doi:10.1089/neu.2006.23.537 (2006).

86 Eddy, N. B. & Leimbach, D. Synthetic analgesics. II. Dithienylbutenyl- and dithienylbutylamines. J Pharmacol Exp Ther 107, 385-393 (1953).

87 Zheng, G. X. et al. Massively parallel digital transcriptional profiling of single cells. Nat Commun 8, 14049, doi:10.1038/ncomms14049 (2017).

88 Hao, Y. et al. Integrated analysis of multimodal single-cell data. Cell 184, 3573-3587 e3529, doi:10.1016/j.ce11.2021.04.048 (2021).

89 Korsunsky, I. et al. Fast, sensitive and accurate integration of single-cell data with Harmony. Nat Methods 16, 1289-1296, doi:10.1038/s41592-019-0619-0 (2019).

90 Love, M. I., Huber, W. & Anders, S. Moderated estimation of fold change and dispersion for RNA-seq data with DESeq2. Genome Biol 15, 550, doi:10.1186/s13059-014-0550-8 (2014).

91 Tan, Y. & Cahan, P. SingleCellNet: A Computational Tool to Classify Single Cell RNA-Seq Data Across Platforms and Across Species. Cell Syst 9, 207-213 e202, doi:10.1016/j.cels.2019.06.004 (2019).

92 Tabula Muris, C. A single-cell transcriptomic atlas characterizes ageing tissues in the mouse. Nature 583, 590-595, doi:10.1038/s41586-020-2496-1 (2020).

93 Street, K. et al. Slingshot: cell lineage and pseudotime inference for single-cell transcriptomics. BMC Genomics 19, 477, doi:10.1186/s12864-018-4772-0 (2018).

94 Efremova, M., Vento-Tormo, M., Teichmann, S. A. & Vento-Tormo, R. CellPhoneDB: inferring cell-cell communication from combined expression of multi-subunit ligand-receptor complexes. Nat Protoc 15, 1484-1506, doi:10.1038/s41596-020-0292-x (2020).

95 Chen, E. Y. et al. Enrichr: interactive and collaborative HTMLS gene list enrichment analysis tool. BMC Bioinformatics 14, 128, doi:10.1186/1471-2105-14-128 (2013).

96 Kuleshov, M. V. et al. Enrichr: a comprehensive gene set enrichment analysis web server 2016 update. Nucleic Acids Res 44, W90-97, doi:10.1093/nar/gkw377 (2016).

What is claimed is:

1. A method for treating, delaying progression of, or reducing the severity of a disorder in a subject, comprising administering to the subject a therapeutic agent capable of hindering and/or inhibiting aberrant cartilage formation and/or osteoclast resorption within joints, muscles, tendons, ligaments, connective tissue, and/or bones experiencing or at risk of experiencing ankylosis, wherein the disorder is characterized with aberrant cartilage formation and/or osteoclast resorption.

2. The method of claim 1, wherein the disorder is one or more of ankylosing spondylitis, heterotopic ossification, rheumatoid arthritis, metastatic bone disease, and Paget disease.

3. The method of claim 1, wherein the subject is a human subject experiencing or at risk of experiencing one or more of ankylosing spondylitis, heterotopic ossification, rheumatoid arthritis, metastatic bone disease, and Paget disease.

4. The method of claim 1, wherein the subject is a human subject having a HLA-B27 mutation within the HLA-B gene.

5. The method of claim 1, wherein the agent is capable of one or more of the following within joints, muscles, tendons, ligaments, connective tissue, and/or bones experiencing or at risk of experiencing ankylosis: inhibition of cartilage formation; inhibition of osteoclast resorption activity; reduction and/or inhibition of TGF-β expression and/or activity; reduction and/or inhibition of pSmad1/5/8 signaling; reduction and/or inhibition of pSmad2/3 signaling; inhibition and/or hindering of chondrogenesis; inhibition and/or hindering of chondrocyte differentiation; prevention ankylosing spondylitis development; and reduction in the severity of ankylosing spondylitis.

6. The method of claim 1, wherein the agent is small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

7. The method of claim 1, wherein the agent is a TGF-β inhibitor or a retinoic acid agonist.

8. The method of claim 7,
wherein the TGF-β inhibitor is selected from antibodies such as (1D11), Fresolimumab, Galunisertib, Lerdelimumab (CAT-152), Metelimumab (CAT-192), GC-1008, SR-2F, and 2G7, small molecule inhibitors such as TβR1I, GW788388 (4-{4-[3-(Pyridin-2-yl)-1H-pyrazol-4-yl]-pyridin-2-yl}-N-(tetrahydro-2H-pyran-4-yl)benzamide hydrate); LY-364947 (4[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxol-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1and Lucanix, and soluble antagonists such as TGFβRII:Fc, and Betaglycan/TGFβRIII;
wherein the retinoic acid agonist is a retinoic acid a agonist selected from ATRA, AM580, AM80 (tamibarotene), BMS753, BD4, AC-93253, AR7, and NRX195183; and
wherein the retinoic acid agonist is a retinoic acid y agonist selected from Palovartene, CD2665, MM11253, 7a, NRX204647.

9. The method of claim 1, wherein the agent is an angiotensin II receptor antagonist.

10. The method of claim 9, wherein the angiotensin II receptor antagonist is selected from losartan and valsartan.

11. A method of treating, delaying progression of, or reducing the severity of spinal cord injuries through administering to a subject having experienced a spinal cord injury a therapeutic agent capable of inhibiting and/or hindering TGF-β activity at and/or around the site of spinal cord injury.

12. The method of claim 11, wherein the administration results in a) inhibiting, delaying progression of, or reducing the severity of fibrotic scar formation at and/or around the site of spinal cord injury; and/or b) inhibiting, delaying progression of, or reducing the severity of fibroblast differentiation of mesenchymal stromal/stem cells (MSCs) at and/or around the site of spinal cord injury.

13. The method of claim 11, wherein the therapeutic agent capable of inhibiting and/or hindering TGF-β activity at and/or around the site of spinal cord injury is a TGF-β inhibitor.

14. The method of claim 13, wherein the TGF-β inhibitor is a small molecule, antibody or functional portion or fragment thereof, protein, peptide, siRNA, antagonist, agonist, compound, or nucleotide construct which either reversibly or irreversibly bind TGF-β and prevent its binding to a TGF-β receptor on a cell or tissue in the subject.

15. The method of claim 13, wherein the TGF-β inhibitor is selected from antibodies such as (1D11), Fresolimumab, Galunisertib, Lerdelimumab (CAT-152), Metelimumab (CAT-192), GC-1008, SR-2F, and 2G7, small molecule inhibitors such as TβR11, GW788388 (4-{4-[3-(Pyridin-2-yl)-1H-pyrazol-4-yl]-pyridin)}-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxol-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1 and Lucanix, and soluble antagonists such as TGFβRH:Fc, and Betaglycan/TGFβRIII.

16. A method of treating, delaying progression of, or reducing the severity of fibrotic scar formation at and/or around a site of spinal cord injury through administering to a subject having experienced a spinal cord injury a therapeutic agent capable of inhibiting and/or hindering TGF-β activity at and/or around the site of spinal cord injury.

17. The method of claim 16, wherein the therapeutic agent capable of inhibiting and/or hindering TGF-β activity at and/or around the site of spinal cord injury is a TGF-β inhibitor.

18. The method of claim 16, wherein the TGF-β inhibitor is a small molecule, antibody or functional portion or fragment thereof, protein, peptide, siRNA, antagonist, agonist, compound, or nucleotide construct which either reversibly or irreversibly bind TGF-β and prevent its binding to a TGF-β receptor on a cell or tissue in the subject.

19. The method of claim 16, wherein the TGF-β inhibitor is selected from antibodies such as (1D11), Fresolimumab, Galunisertib, Lerdelimumab (CAT-152), Metelimumab (CAT-192), GC-1008, SR-2F, and 2G7, small molecule inhibitors such as TβR1I, GW788388 (4-{4-[3-(Pyridin-2-yl)-1H-pyrazol-4-yl]-pyridin-2-yl}-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxol-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1 and Lucanix, and soluble antagonists such as TGFβRH:Fc, and Betaglycan/TGFβRIII.

20. A method of treating, delaying progression of, or reducing the severity of fibroblast differentiation of MSCs at and/or around a site of spinal cord injury through administering to a subject having experienced a spinal cord injury a therapeutic agent capable of inhibiting and/or hindering TGF-β activity at and/or around the site of spinal cord injury.

21. The method of claim 20, wherein the therapeutic agent capable of inhibiting and/or hindering TGF-β activity at and/or around the site of spinal cord injury is a TGF-β inhibitor.

22. The method of claim 20, wherein the TGF-β inhibitor is a small molecule, antibody or functional portion or fragment thereof, protein, peptide, siRNA, antagonist, agonist, compound, or nucleotide construct which either reversibly or irreversibly bind TGF-β and prevent its binding to a TGF-β receptor on a cell or tissue in the subject.

23. The method of claim 22, wherein the TGF-β inhibitor is selected from antibodies such as (1D11), Fresolimumab, Galunisertib, Lerdelimumab (CAT-152), Metelimumab (CAT-192), GC-1008, SR-2F, and 2G7, small molecule inhibitors such as TβR1I, GW788388 (4-{4-[3-(Pyridin-2-yl]-1H-pyrazol-4-yl}-pyrin-N-(tetrahydro-2H-pyran-4-yl)benzamide hydrate); LY-364947 (4-[3-(2-pyridinyl)-1H-pyrazol-4-yl]-quinoline), RepSox (2-[3-(6-Methyl-2-pyridinyl)-1H-pyrazol-4-yl]-1,5-naphthyridine), SB 431542 (4-(5-Benzol[1,3]dioxol-5-yl-4-pyrldin-2-yl-1H-imidazol-2-yl)-benzamide hydrate), LY-550410, LY-580276, LY-2109761, and SX-007, antisense oligonucleotides such as AP-11014, AP-12009, and NovaRx, aptamers such as Trx-xFoxH1b, antisense vaccines such as Trx-Lef1and Lucanix, and soluble antagonists such as TGFβRII:Fc, and Betaglycan/TGFβRIII.
